(19) United States
(12) Patent Application Publication
(10) Pub. No.: US 2023/0190769 A1
(43) Pub. Date: Jun. 22, 2023

(54) E PROTEIN CHANNEL BLOCKERS AND ORF3 INHIBITORS AS ANTI-COVID-19 AGENTS

(71) Applicant: YISSUM RESEARCH DEVELOPMENT COMPANY OF THE HEBREW UNIVERSITY OF JERUSALEM LTD., JERUSALEM (IL)

(72) Inventors: Isaiah ARKIN, Har Adar (IL); Prabhat Pratap SINGH TOMAR, Jerusalem (IL); Miriam KRUGLIAK, Beit El (IL)

(21) Appl. No.: 17/977,349

(22) Filed: Oct. 31, 2022

Related U.S. Application Data
(63) Continuation-in-part of application No. PCT/IL2021/050501, filed on May 2, 2021.
Application No. PCT/IL2021/050501 is a continuation-in-part of application No. PCT/IL2021/051396, filed on Nov. 24, 2021.
(60) Provisional application No. 63/018,598, filed on May 1, 2020.
(60) Provisional application No. 63/117,619, filed on Nov. 24, 2020.
(60) Provisional application No. 63/274,979, filed on Nov. 3, 2021.
(60) Provisional application No. 63/117,619, filed on Nov. 24, 2020.

(51) Int. Cl. A61K 31/565; A61K 31/136; A61K 38/12; A61K 31/506; A61K 31/517; A61P 31/14

(52) U.S. Cl. CPC A61K 31/565; A61K 31/136; A61K 38/12; A61K 31/506; A61K 31/517; A61P 31/14

(57) ABSTRACT

Pharmaceutical compositions comprising a SARS-CoV-2 E protein channel blocker and ORF3 inhibitors for treating or preventing SARS-CoV-2 virulence in a subject, are provided. Further provides is a pharmaceutical composition comprising a SARS-CoV-2 E protein channel blocker or an ORF3 inhibitor for preventing SARS-CoV-2 cell entry, uncoating and/or release from a cell.

### CROSS REFERENCE TO RELATED APPLICATIONS

The present application is a Continuation-in-Part (CIP) of PCT Patent Application No. PCT/IL2021/050501, filed May 2, 2021, which claims the benefit of priority of U.S. Provisional Patent Application No. 63/018,598, titled “E PROTEIN CHANNEL BLOCKERS AS ANTI-COVID-19 AGENTS”, filed May 1, 2020, and of U.S. Provisional Patent Application No. 63/117,619, titled “E PROTEIN CHANNEL BLOCKERS AND ORF3 INHIBITORS AS ANTI-COVID-19 AGENTS”, filed Nov. 24, 2020, the contents of both are incorporated herein by reference in their entirety.

The present application is a Continuation-in-Part (CIP) of PCT Patent Application No. PCT/IL2021/051396, filed Nov. 24, 2021, which claims the benefit of priority of U.S. Provisional Patent Application No. 63/117,619, titled “E PROTEIN CHANNEL BLOCKERS AND ORF3 INHIBITORS AS ANTI-COVID-19 AGENTS”, filed Nov. 24, 2020, and U.S. Provisional Patent Application No. 63/274,979, titled “E PROTEIN CHANNEL BLOCKERS AND ORF3 INHIBITORS AS ANTI-COVID-19 AGENTS”, filed Nov. 3, 2021, the contents of both are incorporated herein by reference in their entirety.

### REFERENCE TO AN ELECTRONIC SEQUENCE LISTING

The contents of the electronic sequence listing (06949-P0047C-HUJI-P-059-US; Size: 3,147 bytes; and Date of Creation: Oct. 31, 2022) is herein incorporated by reference in its entirety.

### FIELD OF INVENTION

The present invention is in the field of anti-viral therapy.

### BACKGROUND

Coronaviruses are positive-sense, single-stranded RNA viruses that are often associated with mild respiratory tract infections in humans. However, three members of the family have received notoriety due to their abnormal virulence: SARS-CoV-1 was the etiological agent of the SARS epidemic in the winter of 2002/3 that caused 774 deaths amongst 8,098 cases; MERS-CoV was responsible for the MERS epidemic that started from 2012 with 862 deaths from 2506 infections; Finally, SARS-CoV-2 is responsible for the ongoing COVID-2019 pandemic resulting in 1.31 million deaths out of 54,068,330 cases (as of Sun Nov. 15, 2020.

Genomic analyses have indicated that SARS-CoV-1 and SARS-CoV-2 are very similar to one another (ca. 80%) but are distinct from most other Coronaviridae members that infect humans. Both viruses have been placed in subgroup B in the Betacoronavirus genus within the Orthocoronavirinae subfamily of the Coronaviridae.

Of all coronavirus' structural proteins, E is the least understood in terms of mechanism of action and structure. Functionally, the E protein has been implicated in viral assembly, release, and pathogenesis. Yet crucially, coronavirus E proteins are important for viral pathogenesis, and attenuated viruses lacking the protein have even been suggested to serve as vaccine candidates.

SARS-CoV-2 3a protein, also known as open reading frame 3a (ORF3a), is implicated in assembly of homotetrameric potassium sensitive ion channels (viroporin) and may modulate virus release. Additionally, it is implicated in pathogenesis, including up-regulation of expression of fibrinogen subunits FGA, FGB and FGG in host lung epithelial cells, inducement of apoptosis in cell culture.

### SUMMARY

According to a first aspect, there is provided a method for treating or preventing SARS-CoV-2 virulence in a subject in need thereof, comprising administering to the subject a therapeutically effective amount of any one of: SARS-CoV-2 E protein channel blocker and a SARS-CoV-2 3a protein inhibitor, thereby treating or preventing SARS-CoV-2 virulence in the subject.

According to another aspect, there is provided a pharmaceutical composition comprising a SARS-CoV-2 E protein channel blocker and/or SARS-CoV-2 3a protein inhibitor for use in the treatment or prevention of SARS-CoV-2 virulence in a subject in need thereof.

In some embodiments, preventing comprises preventing any one of: SARS-CoV-2 entry to a cell of the subject, uncoating of the SARS-CoV-2 in a cell of the subject, release of the SARS-CoV-2 from a cell of the subject, and any combination thereof.

In some embodiments, the subject is infected or suspected of being infected by SARS-CoV-2.

In some embodiments, the SARS-CoV-2 E protein channel blocker is at least one molecule selected from the group consisting of: 5-Azacytidine, Memantine, Gliclazide, Mavorixafor, Saroglitazar Magnesium, Mebrofenin, Cyclen, Kasugamycin, Plerixafor, and any salt thereof.

In some embodiments, the SARS-CoV-2 E protein channel blocker is for use at a daily dose of 0.01 to 500 mg/kg.

In some embodiments, the SARS-CoV-2 E protein channel blocker is Ginsenoside.

In some embodiments, the SARS-CoV-2 E protein channel blocker is Memantine.

In some embodiments, the SARS-CoV-2 3a protein inhibitor is at least one molecule selected from the group consisting of: Capreomycin, Pentamidine, Spectinomycin, Kasugamycin, Plerixafor, Flumatinib, Litronesib, Darapladib, Floxuridine, and Fludarabine.

In some embodiments, the SARS-CoV-2 3a protein inhibitor is Capreomycin.

In some embodiments, the prevention comprises prevention of any one of: SARS-CoV-2 entry to a cell of the subject, uncoating of the SARS-CoV-2, release of the SARS-CoV-2 from a cell of the subject, and any combination thereof.

According to another aspect, there is provided a pharmaceutical composition comprising a SARS-CoV-2 3a protein inhibitor and a SARS-CoV-2 E channel blocker. In some embodiments, the pharmaceutical composition comprises Flumatinib and Darapladib.

According to another aspect, there is provided a combination of a SARS-CoV-2 3a protein inhibitor and a SARS-CoV-2 E channel blocker, for use in the treatment or prevention of SARS-CoV-2 virulence in a subject in need thereof, wherein the SARS-CoV-2 3a protein inhibitor and a SARS-CoV-2 E channel blocker are provided at a molar per molar ratio ranging from 10:1 to 1:10 to the subject.

In some embodiments, the pharmaceutical composition comprises Flumatinib and Darapladib.

According to another aspect, there is provided a method for treating or preventing SARS-CoV-2 virulence in a subject in need thereof, the method comprising administering to the subject a therapeutically effective amount of a pharmaceutical composition comprising a SARS-CoV-2 3a protein inhibitor and a SARS-CoV-2 E channel blocker, thereby treating or preventing SARS-CoV-2 virulence in the subject.

According to another aspect, there is provided a method for treating or preventing SARS-CoV-2 virulence in a subject in need thereof, the method comprising administering to the subject a therapeutically effective amount of a pharmaceutical composition comprising Flumatinib and Darapladib.

In some embodiments, the SARS-CoV-2 protein 3a inhibitor is selected from Flumatinib or Darapladib. In some embodiments, the SARS-CoV-2 E channel blocker is selected from the group consisting of: Mavorixafor, Saroglitazar, Mebrofenin, Cyclen, Plerixafor, and Gliclazide.

In some embodiments, the SARS-CoV-2 protein 3a inhibitor is Flumatinib and the SARS-CoV-2 E channel blocker is selected from the group consisting of: Mavorixafor, Saroglitazar, Mebrofenin, Cyclen, Plerixafor, and Gliclazide.

In some embodiments, the SARS-CoV-2 protein 3a inhibitor is Darapladib and the SARS-CoV-2 E channel blocker is selected from the group consisting of: Mavorixafor, Mebrofenin, and Cyclen.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIGS. 1A-1B include graphs showing membrane permeabilization assay. Growth curves (n=2) of bacteria as a function of SARS-CoV-2 E protein expression (1A, right) or as a function of SARS-CoV-2 3a protein expression (1B). Bacteria that express the maltose binding protein without a conjugated viral ion channel are shown in the left panel as a negative control. Bacteria that express the influenza M2 viroporin, as a positive control, are shown at the centre. Induction at different IPTG concentrations (as noted), takes place when the bacteria density reaches an O.D.600 nm of 0.2. Growth O.D.600 nm values were collected every 15 min. FIG. 1B shows growth curves of bacteria as a function of SARS-CoV-3a protein expression. Negative control (no channel; NC); no drug (ND).

FIGS. 2A-2B include graphs showing K⁺ conductivity assay. Impact of viral protein SARS-CoV-2 E protein on the growth of K⁺-uptake deficient bacteria (left panel, 2A). Different protein expression levels are achieved by varying the concentration of the IPTG inducer, as noted. Bacterial growth rate as a function of [K⁺] is plotted in the right panel (2A). (2B) depicts the impact of SARS CoV-2 3a protein on the growth of K⁺-uptake deficient bacteria, using varying concentration of the IPTG inducer.

FIGS. 3A-3B include graphs showing fluorescence-based H⁺ conductivity assay. The fluorescence of bacteria that harbor pHluorin, a pH-sensitive GFP22, was examined as a function of SARS CoV-2 E protein expression (3A) or SARS CoV-2 3a protein expression (3B). Protein levels were governed by the level of the inducer (IPTG) as indicated. The results are an average of two independent experiments, with standard deviations depicted as error bars.

FIGS. 4A-4B include graphs showing compound screening results using the positive and negative genetic tests. Impact of different drugs, as noted, and E protein expression on the growth rates of bacteria. (4A) Negative genetic test in which SARS-CoV-2 E protein is expressed at an elevated level (40 μM [IPTG]) and is therefore deleterious to bacteria. In this instance inhibitory drugs enhance bacterial growth. (4B) Positive genetic test in which SARS-CoV-2 E protein is expressed at low level (10 μM [IPTG]) in K⁺-uptake deficient bacteria. In this instance inhibitory drugs reduce bacterial growth. In both panels the impact on growth in comparison to growth without any drug is listed.

FIGS. 5A-5C include graphs showing Mavorixafor screening results using the negative assay: Viral channel harmful to bacteria, wherein blocker increases growth (5A), positive assay: Viral channel essential to bacteria, wherein blocker decreases growth (5B), and fluorescence assay: Viral channel alters Fluorescence, wherein blocker decreases fluorescence change (5C) of SARS-CoV-2 E protein expressing bacteria.

FIGS. 6A-6C include graphs showing Saroglitazar magnesium screening results using the negative assay: Viral channel harmful to bacteria, wherein blocker increases growth (6A), positive assay: Viral channel essential to bacteria, wherein blocker decreases growth (6B), and fluorescence assay: Viral channel alters Fluorescence, wherein blocker decreases fluorescence change (6C) of SARS-CoV-2 E protein expressing bacteria.

FIGS. 7A-7C include graphs showing Mebrofenin screening results using the negative assay: Viral channel harmful to bacteria, wherein blocker increases growth (7A), positive assay: Viral channel essential to bacteria, wherein blocker decreases growth (7B), and fluorescence assay: Viral channel alters Fluorescence, wherein blocker decreases fluorescence change (7C) of SARS-CoV-2 E protein expressing bacteria.

FIGS. 8A-8C include graphs showing Cyclen screening results using the negative assay: Viral channel harmful to bacteria, wherein blocker increases growth (8A), positive assay: Viral channel essential to bacteria, wherein blocker decreases growth (8B), and fluorescence assay: Viral channel alters Fluorescence, wherein blocker decreases fluorescence change (8C) of SARS-CoV-2 E protein expressing bacteria.

FIGS. 9A-9C include graphs showing Kasugamycin screening results using the negative assay: Viral channel harmful to bacteria, wherein blocker increases growth (9A), positive assay: Viral channel essential to bacteria, wherein blocker decreases growth (9B), and fluorescence assay: Viral channel alters Fluorescence, wherein blocker decreases fluorescence change (9C) of SARS-CoV-2 E protein expressing bacteria.

FIGS. 10A-10C include graphs showing 5-Azacytidine screening results using the negative assay: Viral channel harmful to bacteria, wherein blocker increases growth (10A), positive assay: Viral channel essential to bacteria, wherein blocker decreases growth (10B), and fluorescence assay: Viral channel alters Fluorescence, wherein blocker decreases fluorescence change (10C) of SARS-CoV-2 E protein expressing bacteria.

FIGS. 11A-11C include graphs showing Plerixafor (octahydrochloride) screening results using the negative assay: Viral channel harmful to bacteria, wherein blocker increases growth (11A), positive assay: Viral channel essential to bacteria, wherein blocker decreases growth (11B), and fluorescence assay: Viral channel alters Fluorescence, wherein blocker decreases fluorescence change (11C) of SARS-CoV-2 E protein expressing bacteria.

FIGS. 12A-12C include graphs showing Plerixafor screening results using the negative assay: Viral channel harmful to bacteria, wherein blocker increases growth (12A), positive assay: Viral channel essential to bacteria, wherein blocker decreases growth (12B), and fluorescence assay: Viral channel alters Fluorescence, wherein blocker decreases fluorescence change (12C) of SARS-CoV-2 E protein expressing bacteria.

FIGS. 13A-13C include graphs showing Capreomycin (sulfate) screening results using the negative assay: Viral channel harmful to bacteria, wherein blocker increases growth (13A), positive assay: Viral channel essential to bacteria, wherein blocker decreases growth (13B), and fluorescence assay: Viral channel alters Fluorescence, wherein blocker decreases fluorescence change (13C) of SARS-CoV-2 E protein expressing bacteria.

FIGS. 14A-14C include graphs showing Pentamidine (isethionate) screening results using the negative assay: Viral channel harmful to bacteria, wherein blocker increases growth (14A), positive assay: Viral channel essential to bacteria, wherein blocker decreases growth (14B), and fluorescence assay: Viral channel alters Fluorescence, wherein blocker decreases fluorescence change (14C) of SARS-CoV-2 3a protein expressing bacteria.

FIGS. 15A-15C include graphs showing Spectinomycin (dihydrochloride) screening results using the negative assay: Viral channel harmful to bacteria, wherein blocker increases growth (15A), positive assay: Viral channel essential to bacteria, wherein blocker decreases growth (15B), and fluorescence assay: Viral channel alters Fluorescence, wherein blocker decreases fluorescence change (15C) of SARS-CoV-2 3a protein expressing bacteria.

FIGS. 16A-16C include graphs showing Kasugamycin (hydrochloride hydrate) screening results using the negative assay: Viral channel harmful to bacteria, wherein blocker increases growth (16A), positive assay: Viral channel essential to bacteria, wherein blocker decreases growth (16B), and fluorescence assay: Viral channel alters Fluorescence, wherein blocker decreases fluorescence change (16C) of SARS-CoV-2 3a protein expressing bacteria.

FIGS. 17A-17C include graphs showing Plerixafor screening results using the negative assay: Viral channel harmful to bacteria, wherein blocker increases growth (17A), positive assay: Viral channel essential to bacteria, wherein blocker decreases growth (17B), and fluorescence assay: Viral channel alters Fluorescence, wherein blocker decreases fluorescence change (17C) of SARS-CoV-2 3a protein expressing bacteria.

FIGS. 18A-18C include graphs showing Flumatinib screening results using the negative assay: Viral channel harmful to bacteria, wherein blocker increases growth (18A), positive assay: Viral channel essential to bacteria, wherein blocker decreases growth (18B), and fluorescence assay: Viral channel alters Fluorescence, wherein blocker decreases fluorescence change (18C) of SARS-CoV-2 3a protein expressing bacteria.

FIGS. 19A-19C include graphs showing Litronesib screening results using the negative assay: Viral channel harmful to bacteria, wherein blocker increases growth (19A), positive assay: Viral channel essential to bacteria, wherein blocker decreases growth (19B), and fluorescence assay: Viral channel alters Fluorescence, wherein blocker decreases fluorescence change (19C) of SARS-CoV-2 3a protein expressing bacteria.

FIGS. 20A-20C include graphs showing Darapladib screening results using the negative assay: Viral channel harmful to bacteria, wherein blocker increases growth (20A), positive assay: Viral channel essential to bacteria, wherein blocker decreases growth (20B), and fluorescence assay: Viral channel alters Fluorescence, wherein blocker decreases fluorescence change (20C) of SARS-CoV-2 3a protein expressing bacteria.

FIGS. 21A-21C include graphs showing Floxuridine screening results using the negative assay: Viral channel harmful to bacteria, wherein blocker increases growth (21A), positive assay: Viral channel essential to bacteria, wherein blocker decreases growth (21B), and fluorescence assay: Viral channel alters Fluorescence, wherein blocker decreases fluorescence change (21C) of SARS-CoV-2 3a protein expressing bacteria.

FIGS. 22A-22C include graphs showing Fludarabine screening results using the negative assay: Viral channel harmful to bacteria, wherein blocker increases growth (22A), positive assay: Viral channel essential to bacteria, wherein blocker decreases growth (22B), and fluorescence assay: Viral channel alters Fluorescence, wherein blocker decreases fluorescence change (22C) of SARS-CoV-2 3a protein expressing bacteria.

FIG. 23 includes a vertical bar graph showing the effect of various tested drugs on the viability of Vero-E6 cells which were infected with SARS-CoV-2 at a multiplicity of infection (MOI) of 0.01.

FIG. 24 includes a graph showing the effect of individual 3a protein inhibitors on cell survival. Both Flumatinib and Darapladib provided near full protection at a concentration of 3 μM.

FIGS. 25A-25C include graphs showing the effect of the combination of a 3a protein inhibitor and various E protein inhibitors on cell survival. (25A) Combination of Flumatinib and Mavorixafor; (25B) Combination of Flumatinib and Cyclen; and (25C) Combination of Flumatinib and Sargolitazar.

FIGS. 26A-26C include graphs showing the effect of a combination of 3a protein inhibitors on cell survival. Combinations of Flumatinib and Darapladib were tested. (26A) Flumatinib (1 μM) and Darapladib (0.3 μM); (26B) Flumatinib (0.1 μM) and Darapladib (1 μM); and (26C) Flumatinib (0.3 μM) and Darapladib (1 μM).

FIGS. 27A-27C include graphs showing the effect of 3a inhibitors, E protein blockers, or both, on cell survival. (27A) The sole effect of 3 μM of either 3a inhibitors or E protein blockers; (27B) Combinations of 304 of the E protein blockers: Mavorixafor (Mayo), Saroglitazar (Saro), Mebrofenin (Mebro), Cyclen, 5-Azacytidine (5-aza), Plerixafor (Plerixa), Kasugamycin (Kasuga), and Gliclazide (glicla), with the 3a protein inhibitors: Capreomycin (Capero), Pentamidine (Penta), Spectinomycin (Spectino), and Flumatinib (Fluma); (27C) Combinations of 3 μM of the E protein blockers: Mayo, Saro, Mebro, Cyclen, 5-aza, Plerixa, Kasuga, and glicla, with the 3a protein inhibitors: Fludarabine (Fluda), Litronesib (Litro), Darapladib (Darap), and Floxuridine (Fluxo).

### DETAILED DESCRIPTION

The present invention, in some embodiments, provides compositions comprising a SARS-CoV-2 E protein channel blocker and/or a SARS-CoV-2 3a protein inhibitor for treating or preventing SARS-CoV-2 virulence in a subject. The present invention, in some embodiments, provides compositions comprising a SARS-CoV-2 E protein channel blocker and/or a SARS-CoV-2 3a protein inhibitor, for preventing SARS-CoV-2 2 cell entry, uncoating and/or release from a cell.

### SARS-CoV-2 E Protein Channel Blockers

The invention is based, at least in part, on the finding using three bacteria-based assays, that SARS-CoV-2 E protein is an ion channel. The invention is further based, at least in part, on a finding that Gliclazide, Memantine, Mavorixafor, Saroglitazar Magnesium, Mebrofenin, Cyclen, Kasugamycin, Azacytidine, and Plerixafor, inhibit SARS-CoV-2 E protein and therefore can be used to treat and prevent SARS-CoV-2 virulence.

SARS-CoV-2 E protein is known to one skilled in the art and has a GenBank Accession no: QIH45055.1. According to some embodiments, the SARS-CoV-2 E protein comprises the amino acid sequence as set forth in SEQ ID NO 1: MYSFVSEETGTLIVNSVLLFLAFVVFLLVTLAILTALRLCAYCCNIVNVSLVKPSFY VYSRVKNLNSSRVPDLLV. According to some embodiments, the SARS-CoV-2 E protein comprises an analog of SEQ ID NO: 1, such as an analog having at least 85%, at least 90%, at least 95% identity to SEQ ID NO: 1.

According to some embodiments, the invention provides a method of treating or preventing SARS-CoV-2 virulence in a subject in need thereof, comprising administering to the subject a therapeutically effective amount of a SARS-CoV-2 E protein channel blocker, thereby treating or preventing SARS-CoV-2 virulence in the subject.

In some embodiments, the invention provides a method of treating or preventing Coronavirus disease 2019 (COVID-19) in a subject in need thereof, comprising administering to the subject a therapeutically effective amount of a SARS-CoV-2 E protein channel blocker, thereby treating or preventing COVID-19.

According to some embodiments, the invention provides a method of preventing SARS-CoV-2 release from a cell. In some embodiments, the method comprises contacting a cell with a SARS-CoV-2 E protein channel blocker, thereby preventing SARS-CoV-2 release from the cell.

According to some embodiments, the invention provides a method of preventing SARS-CoV-2 cell entry. In some embodiments, the method comprises contacting a cell with a SARS-CoV-2 E protein channel blocker, thereby preventing SARS-CoV-2 cell entry.

According to some embodiments, the invention provides a method of preventing SARS-CoV-2 uncoating. In some embodiments, the method comprises contacting a cell with a SARS-CoV-2 E protein channel blocker, thereby preventing SARS-CoV-2 uncoating.

According to some embodiments, a cell is a cell of a subject. According to some embodiments, contacting comprises administering to the subject. According to some embodiments, the subject is a subject infected or suspected as being infected by SARS-CoV-2.

According to some embodiments, there is provided a method for treating or preventing SARS-CoV-2 virulence in a subject in need thereof, comprising administering to the subject a therapeutically effective amount of 5-Azacytidine, thereby treating or preventing SARS-CoV-2 virulence in said subject.

According to some embodiments, there is provided a pharmaceutical composition comprising 5-Azacytidine, for use in the treatment and/or prevention of SARS-CoV-2 virulence in a subject in need thereof.

According to some embodiments, the SARS-CoV-2 E protein channel blocker is at least one molecule selected from: Memantine, Gliclazide, Mavorixafor, Saroglitazar Magnesium, Mebrofenin, Cyclen, Kasugamycin, Azacytidine, Plerixafor, or any salt thereof.

According to some embodiments, the invention provides a SARS-CoV-2 E protein channel blocker for use in treating or preventing SARS-CoV-2 virulence in a subject in need thereof.

According to some embodiments, the invention provides a SARS-CoV-2 E protein channel blocker for use in preventing SARS-CoV-2 release from a cell.

According to some embodiments, the SARS-CoV-2 E protein channel blocker is within a pharmaceutical composition. In some embodiments, the pharmaceutical composition further comprises a pharmaceutically acceptable carrier.

According to some embodiments, the invention provides a pharmaceutical composition comprising Azacytidine, an analog or a salt thereof, for treating a viral infection.

According to some embodiments, the SARS-CoV-2 E protein channel blocker is Azacytidine, an analog or a salt thereof. According to some embodiments, the SARS-CoV-2 E protein channel blocker is 5-Azacytidine.

Azacytidine, as used herein, includes Azacytidine (CAS: 320-67-2; 4-Amino-1-β-D-ribofuranosyl-s-triazin-2(1H)-one), as well as pharmaceutically acceptable salts, solvates, hydrates, or mixtures thereof. Azacytidine is described, for example in WO2012135405A1. The terms “5-Azacytidine” and “Azacytidine” are used herein interchangeably.

According to some embodiments, the invention provides a pharmaceutical composition comprising Memantine, an analog or a salt thereof, for use in the treatment of a viral infection. In some embodiments, the viral infection comprises a coronaviruses infection. In some embodiments, the viral infection comprises an infection by virus having an E protein being an ion channel. In some embodiments, the viral infection is a coronaviruses infection.

According to some embodiments, the SARS-CoV-2 E protein channel blocker is Memantine, an analog or a salt thereof. According to some embodiments, the SARS-CoV-2 E protein channel blocker is memantine hydrochloride.

Memantine, as used herein, includes memantine (CAS: 19982-08-2; 1-amino-3,5-dimethyladamantane), as well as pharmaceutically acceptable salts, solvates, hydrates, or mixtures thereof. Memantine is described, for example, in U.S. Pat. Nos. 3,391,142, 5,891,885, 5,919,826, and 6,187,338.

According to some embodiments, the invention provides a pharmaceutical composition comprising Gliclazide, an analog or a salt thereof, for treating a viral infection. In some embodiments, the viral infection is an infection by virus having an E protein being an ion channel.

According to some embodiments, the SARS-CoV-2 E protein channel blocker is Gliclazide an analog or a salt thereof.

Gliclazide, as used herein, includes gliclazide (CAS: 21187-98-4; 1-(3-azabicyclo(3.3.0)oct-3-yl)-3-(p-tolylsulfonyl)urea) as well as pharmaceutically acceptable salts, solvates, hydrates, or mixtures thereof.

According to some embodiments, the SARS-CoV-2 E protein channel blocker is selected from a group including Ginsenoside.

According to some embodiments, the invention provides a pharmaceutical composition comprising Mavorixafor, an analog or a salt thereof, for treating a viral infection.

According to some embodiments, the SARS-CoV-2 E protein channel blocker is Mavorixafor, an analog or a salt thereof. According to some embodiments, the SARS-CoV-2 E protein channel blocker is Mavorixafor.

Mavorixafor, as used herein, includes Mavorixafor (CAS: 558447-26-0; N-(1H-benzimidazol-2-ylmethyl)-N-[(8S)-5,6,7,8-tetrahydroquinolin-8-yl]butane-1,4-diamine), as well as pharmaceutically acceptable salts, solvates, hydrates, or mixtures thereof. Mavorixafor is described, for example, in U.S. Pat. No. 7,332,605, and as compound 89 from a series of 169 analogues in WO2003055876.

According to some embodiments, the invention provides a pharmaceutical composition comprising Saroglitazar, an analog or a salt thereof, for treating a viral infection.

According to some embodiments, the SARS-CoV-2 E protein channel blocker is Saroglitazar, an analog or a salt thereof. According to some embodiments, the SARS-CoV-2 E protein channel blocker is Saroglitazar Magnesium.

Saroglitazar, as used herein, includes Saroglitazar (CAS: 495399-09-2; (αS)-α-Ethoxy-4-[2-[2-methyl-5-[4-(methylthio)phenyl]-1H-pyrrol-1-yl] ethoxy]benzenepropanoic Acid), as well as pharmaceutically acceptable salts, solvates, hydrates, or mixtures thereof. Saroglitazar is described, for example, in WO2016181409.

According to some embodiments, the invention provides a pharmaceutical composition comprising Mebrofenin, an analog or a salt thereof, for treating a viral infection.

According to some embodiments, the SARS-CoV-2 E protein channel blocker is Mebrofenin, an analog or a salt thereof. According to some embodiments, the SARS-CoV-2 E protein channel blocker is Mebrofenin.

Mebrofenin, as used herein, includes Mebrofenin (CAS: 78266-06-5; 2-[[2-(3-bromo-2,4,6-trimethylanilino)-2-oxoethyl]-(carboxymethyl)amino]acetic acid), as well as pharmaceutically acceptable salts, solvates, hydrates, or mixtures thereof. Mebrofenin is described, for example, in U.S. Pat. No. 9,878,984.

According to some embodiments, the invention provides a pharmaceutical composition comprising Cyclen, an analog or a salt thereof, for treating a viral infection.

According to some embodiments, the SARS-CoV-2 E protein channel blocker is Cyclen, an analog or a salt thereof. According to some embodiments, the SARS-CoV-2 E protein channel blocker is Cyclen.

Cyclen, as used herein, includes Cyclen (CAS: 294-90-6; 1,4,7,10-Tetraazacyclododecane), as well as pharmaceutically acceptable salts, solvates, hydrates, or mixtures thereof. Cyclen is described, for example in U.S. Pat. No. 9,421,223B2.

According to some embodiments, the invention provides a pharmaceutical composition comprising Kasugamycin, an analog or a salt thereof, for treating a viral infection.

According to some embodiments, the SARS-CoV-2 E protein channel blocker is Kasugamycin, an analog or a salt thereof. According to some embodiments, the SARS-CoV-2 E protein channel blocker is Kasugamycin hydrochloride hydrate (CAS: 19408-46-9).

Kasugamycin, as used herein, includes Kasugamycin (CAS: 6980-18-3; 2-amino-2-[(2R,3 S,5 S,6R)-5-amino-2-methyl-6-[(2R,3 S,5S,6S)-2,3,4,5,6-pentahydroxycyclohexyl]oxyoxan-3-yl]iminoacetic acid), as well as pharmaceutically acceptable salts, solvates, hydrates, or mixtures thereof. Kasugamycin is described, for example in U.S. Pat. No. 3,358,001A.

According to some embodiments, the invention provides a pharmaceutical composition comprising Plerixafor, an analog or a salt thereof, for treating a viral infection.

According to some embodiments, the SARS-CoV-2 E protein channel blocker is Plerixafor, an analog or a salt thereof. According to some embodiments, the SARS-CoV-2 E protein channel blocker is Plerixafor octahydrochloride.

Plerixafor, as used herein, includes Plerixafor (CAS: 155148-31-5; 1-[[4-(1,4,8,11-tetrazacyclotetradec-1-ylmethyl)phenyl]methyl]-1,4,8,11-tetrazacyclotetradecane), as well as pharmaceutically acceptable salts, solvates, hydrates, or mixtures thereof. Plerixafor is described, for example in WO2014125499A1.

### SARS-CoV-2 3a Protein Inhibitor

The invention is based, at least in part, on the finding using three bacteria-based assays, that SARS-CoV-2 3a protein inhibitors can serve as effective agents for treating and preventing SARS-CoV-2 virulence.

SARS-CoV-2 3a protein, also known as open reading frame 3a (ORF3a), is known to one skilled in the art and has a UniProt Accession no: PODTC3.

The terms “3a protein” and “ORF3a” are used herein interchangeably.

According to some embodiments, the SARS-CoV-2 3a protein comprises the amino acid sequence as set forth in SEQ ID NO 2: MDLFIVIRIFTIGTVTLKQGEIKDATPSDFVRATATIPIQASLPFGWLIVGVALLAVFQ SASKIITLKKRWQLALSKGVHFVCNLLLLFVTVYSHLLLVAAGLEAPFLYLYALVY FLQSINFVRIIIVIRLWLCWKCRSKNPLLYDANYFLCWHTNCYDYCIPYNSVTSSIVIT SGDGTTSPISEHDYQIGGYTEKWESGVKDCVVLHSYFTSDYYQLYSTQLSTDTGVE HVTFFIYNKIVDEPEEHVQIHTIDGSSGVVNPVMEPIYDEPTTTTSVPL According to some embodiments, the SARS-CoV-2 3a protein comprises an analog of SEQ ID NO: 2, such as an analog having at least 85%, at least 90%, at least 95% identity to SEQ ID NO: 2.

According to some embodiments, the invention provides a method of treating or preventing SARS-CoV-2 virulence in a subject in need thereof, comprising administering to the subject a therapeutically effective amount of a SARS-CoV-2 3a protein inhibitor, thereby treating or preventing SARS-CoV-2 virulence in the subject.

In some embodiments, the invention provides a method of treating or preventing Coronavirus disease 2019 (COVID-19) in a subject in need thereof, comprising administering to the subject a therapeutically effective amount of a SARS-CoV-2 3a protein inhibitor, thereby treating or preventing COVID-19.

According to some embodiments, the invention provides a method of preventing SARS-CoV-2 release from a cell, the method comprising contacting the cell with a SARS-CoV-2 3a protein inhibitor, thereby preventing SARS-CoV-2 release from the cell.

According to some embodiments, the method comprising contacting the cell with a SARS-CoV-2 3a protein inhibitor, thereby preventing SARS-CoV-2 cell entry.

In some embodiments, the method comprising contacting the cell with a SARS-CoV-2 3a protein inhibitor, thereby preventing SARS-CoV-2 uncoating.

According to some embodiments, the subject is a subject infected or suspected as being infected by SARS-CoV-2.

According to some embodiments, the SARS-CoV-2 3a protein inhibitor is at least one molecule selected from: Capreomycin, Pentamidine, Spectinomycin, Kasugamycin, Plerixafor, Flumatinib, Litronesib, Darapladib, Floxuridine, Fludarabine, or salts thereof.

According to some embodiments, the invention provides a SARS-CoV-2 3a protein inhibitor for use in the treatment or prevention of SARS-CoV-2 virulence, in a subject in need thereof.

According to some embodiments, the invention provides a SARS-CoV-2 3a protein inhibitor for use in the prevention of SARS-CoV-2 release from a cell.

According to some embodiments, the SARS-CoV-2 3a protein inhibitor is within a pharmaceutical composition.

In some embodiments, the viral infection is an infection by virus having a 3a protein.

According to some embodiments, the invention provides a pharmaceutical composition comprising Capreomycin, an analog or a salt thereof, for treating a viral infection.

According to some embodiments, the SARS-CoV-2 3a protein inhibitor is Capreomycin, an analog or a salt thereof. According to some embodiments, the SARS-CoV-2 3a protein inhibitor is Capreomycin sulfate.

Capreomycin, as used herein, includes Capreomycin (CAS: 11003-38-6; IUPAC: (3S)-3,6-diamino-N-[[(2S,5S,8E,11S,15 S)-15-amino-11-[(4R)-2-amino-3,4,5,6-tetrahydropyrimidin-4-yl]-8-[(carbamoylamino)methylidene]-2-(hydroxymethyl)-3,6,9,12,16-pentaoxo-1,4,7,10,13-pentazacyclohexadec-5-yl]methyl]hexanamide; (3 S)-3,6-diamino-N-[[(2S,5S,8E,11S,15S)-15-amino-11-[(4R)-2-amino-3,4,5,6-tetrahydropyrimidin-4-yl]-8-[(carbamoylamino)methylidene]-2-methyl-3,6,9,12,16-pentaoxo-1,4,7,10,13-pentazacyclohexadec-5-yl]methyl]hexanamide), as well as pharmaceutically acceptable salts, solvates, hydrates, or mixtures thereof.

According to some embodiments, the invention provides a pharmaceutical composition comprising Pentamidine, an analog or a salt thereof, for treating a viral infection.

According to some embodiments, the SARS-CoV-2 E protein channel blocker is Pentamidine, an analog or a salt thereof. According to some embodiments, the SARS-CoV-2 3a protein inhibitor is Pentamidine isethionate.

Pentamidine, as used herein, includes Pentamidine (CAS: 100-33-4; IUPAC: 4,4′-[pentane-1,5-diylbis(oxy)]dibenzenecarboximidamide), as well as pharmaceutically acceptable salts, solvates, hydrates, or mixtures thereof.

According to some embodiments, the invention provides a pharmaceutical composition comprising Spectinomycin, an analog or a salt thereof, for treating a viral infection.

According to some embodiments, the SARS-CoV-2 E protein channel blocker is Spectinomycin, an analog or a salt thereof. According to some embodiments, the SARS-CoV-2 3a protein inhibitor is Spectinomycin dihydrochloride.

Spectinomycin, as used herein, includes Spectinomycin (CAS: 1695-77-8; IUPAC: 1R,3S,5R,8R,10S,11S,12S,13R,14S)-8,12,14-trihydroxy-5-methyl-11,13-bis(methylamino)-2,4,9-trioxatricyclo[8.4.0.03,8]tetradecan-7-one), as well as pharmaceutically acceptable salts, solvates, hydrates, or mixtures thereof.

According to some embodiments, the invention provides a pharmaceutical composition comprising Kasugamycin, an analog or a salt thereof, for treating a viral infection.

According to some embodiments, the SARS-CoV-2 3a protein inhibitor is Kasugamycin, an analog or a salt thereof. According to some embodiments, the SARS-CoV-2 3a protein inhibitor is Kasugamycin hydrochloride hydrate.

Kasugamycin, as used herein, includes Kasugamycin (CAS:6980-18-3; IUPAC: 2-amino-2-[(2R,3 S,5S,6R)-5-amino-2-methyl-6-[(2R,3 S,5S,6S)-2,3,4,5,6-pentahydroxycyclohexyl]oxyoxan-3-yl]iminoacetic acid), as well as pharmaceutically acceptable salts, solvates, hydrates, or mixtures thereof.

According to some embodiments, the invention provides a pharmaceutical composition comprising Plerixafor, an analog or a salt thereof, for treating a viral infection.

According to some embodiments, the SARS-CoV-2 3a protein inhibitor is Plerixafor, an analog or a salt thereof. According to some embodiments, the SARS-CoV-2 3a protein inhibitor is Plerixafor.

Plerixafor, as used herein, includes Plerixafor (CAS: 155148-31-5; IUPAC: 1,1′-(1,4-phenylenebismethylene)bis(1,4,8,11-tetraazacyclotetradecane)), as well as pharmaceutically acceptable salts, solvates, hydrates, or mixtures thereof.

According to some embodiments, the invention provides a pharmaceutical composition comprising Flumatinib, an analog or a salt thereof, for treating a viral infection.

According to some embodiments, the SARS-CoV-2 3a protein inhibitor is Flumatinib, an analog or a salt thereof. According to some embodiments, the SARS-CoV-2 3a protein inhibitor is Flumatinib.

Flumatinib, as used herein, includes Flumatinib (CAS: 895519-90-1; IUPAC: 4-[(4-methylpiperazin-1-yl)methyl]-N-[6-methyl-5-[(4-pyridin-3-ylpyrimidin-2-yl)amino]pyridin-3-yl]-3-(trifluoromethyl)benzamide), as well as pharmaceutically acceptable salts, solvates, hydrates, or mixtures thereof.

According to some embodiments, the invention provides a pharmaceutical composition comprising Litronesib, an analog or a salt thereof, for treating a viral infection.

According to some embodiments, the SARS-CoV-2 3a protein inhibitor is Litronesib, an analog or a salt thereof. According to some embodiments, the SARS-CoV-2 3a protein inhibitor is Litronesib.

Litronesib, as used herein, includes Litronesib (CAS: 910634-41-2; IUPAC: N-[(5R)-4-(2,2-dimethylpropanoyl)-5-[[2-(ethylamino)ethyl sulfonylamino]methyl]-5-phenyl-1,3,4-thiadiazol-2-yl]-2,2-dimethylpropanamide), as well as pharmaceutically acceptable salts, solvates, hydrates, or mixtures thereof.

According to some embodiments, the invention provides a pharmaceutical composition comprising Darapladib, an analog or a salt thereof, for treating a viral infection.

According to some embodiments, the SARS-CoV-2 3a protein inhibitor is Darapladib, an analog or a salt thereof. According to some embodiments, the SARS-CoV-2 3a protein inhibitor is Darapladib.

Darapladib, as used herein, includes Darapladib (CAS: 356057-34-6; IUPAC: N-(2-Diethylaminoethyl)-2-[2-[(4-fluorophenyl)methyl sulfanyl]-4-oxo-6,7-dihydro-5H-cyclopenta[d]pyrimidin-1-yl]-N-[[4-[4-(trifluoromethyl)phenyl]phenyl]methyl]acetamide), as well as pharmaceutically acceptable salts, solvates, hydrates, or mixtures thereof.

According to some embodiments, the invention provides a pharmaceutical composition comprising Floxuridine, an analog or a salt thereof, for treating a viral infection.

According to some embodiments, the SARS-CoV-2 3a protein inhibitor is Floxuridine, an analog or a salt thereof. According to some embodiments, the SARS-CoV-2 3a protein inhibitor is Floxuridine.

Floxuridine, as used herein, includes Floxuridine (CAS: 50-91-9; IUPAC: 5-Fluoro-1-[4-hydroxy-5-(hydroxymethyl)tetrahydrofuran-2-yl]-1H-pyrimidine-2,4-dione), as well as pharmaceutically acceptable salts, solvates, hydrates, or mixtures thereof.

According to some embodiments, the invention provides a pharmaceutical composition comprising Fludarabine, an analog or a salt thereof, for treating a viral infection.

According to some embodiments, the SARS-CoV-2 3a protein inhibitor is Fludarabine, an analog or a salt thereof. According to some embodiments, the SARS-CoV-2 3a protein inhibitor is Fludarabine.

Fludarabine, as used herein, includes Fludarabine (CAS: 21679-14-1; IUPAC: [(2R,3 S,4S,5R)-5-(6-amino-2-fluoro-purin-9-yl)-3,4-dihydroxy-oxolan-2-yl]methoxyphosphonic acid), as well as pharmaceutically acceptable salts, solvates, hydrates, or mixtures thereof.

### Pharmaceutical Compositions

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

As used herein, the term “prevention” of a disease, disorder, or condition encompasses the delay, prevention, suppression, or inhibition of the onset of a disease, disorder, or condition. As used in accordance with the presently described subject matter, the term “prevention” relates to a process of prophylaxis in which a subject is exposed to the presently described compositions or formulations prior to the induction or onset of the disease/disorder process. The term “suppression” is used to describe a condition wherein the disease/disorder process has already begun but obvious symptoms of the condition have yet to be realized. Thus, the cells of an individual may have the disease/disorder, but no outside signs of the disease/disorder have yet been clinically recognized. In either case, the term prophylaxis can be applied to encompass both prevention and suppression. Conversely, the term “treatment” refers to the clinical application of active agents to combat an already existing condition whose clinical presentation has already been realized in a patient.

In some embodiments, preventing comprises reducing the disease severity, delaying the disease onset, reducing the disease cumulative incidence, or any combination thereof.

As used herein, the terms “administering,” “administration,” and like terms refer to any method which, in sound medical practice, delivers a composition containing an active agent to a subject in such a manner as to provide a therapeutic effect.

As used herein, the terms “subject” or “individual” or “animal” or “patient” or “mammal,” refers to any subject, particularly a mammalian subject, for whom therapy is desired, for example, a human.

According to some embodiments, the invention provides a method of treating or preventing SARS-CoV-2 virulence in a subject in need thereof, comprising administering to the subject a therapeutically effective amount of a SARS-CoV-2 E protein channel blocker and a SARS-CoV-2 3a protein inhibitor, thereby treating or preventing SARS-CoV-2 virulence in the subject.

In some embodiments, administering comprises providing a SARS-CoV-2 E protein channel blocker and a SARS-CoV-2 3a protein in a therapeutically effective amount, synergistically effective amount, or both, to the subject.

In some embodiments, administering comprises providing at least two SARS-CoV-2 3a protein inhibitors in a therapeutically effective amount, synergistically effective amount, or both, to the subject.

As used herein, the term “synergistically effective amount” comprises any weight or concentration of a SARS-CoV-2 E protein channel blocker and a SARS-CoV-2 3a protein inhibitor, as long as their molar ratio ranges from: 100:1 to 1:100 (M:M), 50:1 to 1:50 (M:M), 30:1 to 1:30 (M:M), 10:1 to 1:10 (M:M), 8:1 to 1:8 (M:M), 5:1 to 1:5 (M:M), 4:1 to 1:4 (M:M), 3:1 to 1:3 (M:M), 2:1 to 1:2 (M:M), or is 1:1 (M:M). Each possibility represents a separate embodiment of the invention.

As used herein, the term “synergistically effective amount” comprises any weight or concentration of a first SARS-CoV-2 3a protein channel inhibitor and a second SARS-CoV-2 3a protein inhibitor, as long as their molar ratio ranges from: 100:1 to 1:100 (M:M), 50:1 to 1:50 (M:M), 30:1 to 1:30 (M:M), 10:1 to 1:10 (M:M), 8:1 to 1:8 (M:M), 5:1 to 1:5 (M:M), 4:1 to 1:4 (M:M), 3:1 to 1:3 (M:M), 2:1 to 1:2 (M:M), or is 1:1 (M:M). Each possibility represents a separate embodiment of the invention.

In some embodiments, the first 3a protein inhibitor comprises any one of Flumatinib and Darapladib. In some embodiments, the second 3a protein inhibitor comprises any one of Flumatinib and Darapladib.

According to some embodiments, there is provided a method for treating or preventing SARS-CoV-2 virulence in a subject in need thereof, the method comprising administering to the subject a therapeutically effective amount of a pharmaceutical composition comprising a SARS-CoV-2 3a protein inhibitor and a SARS-CoV-2 E channel blocker in a molar per molar ratio ranging from 100:1 to 1:100, 50:1 to 1:50, 30:1 to 1:30, 10:1 to 1:10, 8:1 to 1:8, 5:1 to 1:5, 4:1 to 1:4, 3:1 to 1:3, 2:1 to 1:2, or is 1:1, thereby treating or preventing SARS-CoV-2 virulence in the subject. Each possibility represents a separate embodiment of the invention.

In some embodiments, the SARS-CoV-2 protein 3a inhibitor is Flumatinib or Darapladib.

In some embodiments, the SARS-CoV-2 E channel blocker is selected: Mavorixafor, Saroglitazar, Mebrofenin, Cyclen, Plerixafor, and Gliclazide.

In some embodiments, the SARS-CoV-2 protein 3a inhibitor is Flumatinib and the SARS-CoV-2 E channel blocker is selected from: Mavorixafor, Saroglitazar, Mebrofenin, Cyclen, Plerixafor, and Gliclazide.

In some embodiments, the SARS-CoV-2 protein 3a inhibitor is in some embodiments, the SARS-CoV-2 protein 3a inhibitor is Flumatinib or Darapladib and the SARS-CoV-2 E channel blocker is selected from: Mavorixafor, Cyclen, or Mebrofenin.

In some embodiments, the method comprises administering to the subject a therapeutically effective amount of a pharmaceutical composition comprising Flumatinib and Mavorixafor, thereby treating or preventing SARS-CoV-2 virulence in the subject. Each possibility represents a separate embodiment of the invention.

In some embodiments, the method comprises administering to the subject a synergistically effective amount of a pharmaceutical composition comprising Flumatinib and Mavorixafor, thereby treating or preventing SARS-CoV-2 virulence in the subject. Each possibility represents a separate embodiment of the invention.

In some embodiments, the method comprises administering to the subject a pharmaceutical composition comprising Flumatinib and Mavorixafor in a molar per molar ratio ranging from 100:1 to 1:100, 50:1 to 1:50, 30:1 to 1:30, 15:1 to 1:15, 10:1 to 1:10, 8:1 to 1:8, 4:1 to 1:4, 3:1 to 1:3, 2:1 to 1:2, or is 1:1, thereby treating or preventing SARS-CoV-2 virulence in the subject. Each possibility represents a separate embodiment of the invention.

In some embodiments, the method comprises administering to the subject a therapeutically effective amount of a pharmaceutical composition comprising Flumatinib and Cyclen, thereby treating or preventing SARS-CoV-2 virulence in the subject. Each possibility represents a separate embodiment of the invention.

In some embodiments, the method comprises administering to the subject a synergistically effective amount of a pharmaceutical composition comprising Flumatinib and Cyclen, thereby treating or preventing SARS-CoV-2 virulence in the subject. Each possibility represents a separate embodiment of the invention.

In some embodiments, the method comprises administering to the subject a pharmaceutical composition comprising Flumatinib and Cyclen in a molar per molar ratio ranging from 100:1 to 1:100, 50:1 to 1:50, 30:1 to 1:30, 15:1 to 1:15, 10:1 to 1:10, 8:1 to 1:8, 4:1 to 1:4, 3:1 to 1:3, 2:1 to 1:2, or is 1:1, thereby treating or preventing SARS-CoV-2 virulence in the subject. Each possibility represents a separate embodiment of the invention.

In some embodiments, the method comprises administering to the subject a therapeutically effective amount of a pharmaceutical composition comprising Flumatinib and Mebrofenin, thereby treating or preventing SARS-CoV-2 virulence in the subject. Each possibility represents a separate embodiment of the invention.

In some embodiments, the method comprises administering to the subject a synergistically effective amount of a pharmaceutical composition comprising Flumatinib and Mebrofenin, thereby treating or preventing SARS-CoV-2 virulence in the subject. Each possibility represents a separate embodiment of the invention.

In some embodiments, the method comprises administering to the subject a pharmaceutical composition comprising Flumatinib and Mebrofenin in a molar per molar ratio ranging from 100:1 to 1:100, 50:1 to 1:50, 30:1 to 1:30, 15:1 to 1:15, 10:1 to 1:10, 8:1 to 1:8, 4:1 to 1:4, 3:1 to 1:3, 2:1 to 1:2, or is 1:1, thereby treating or preventing SARS-CoV-2 virulence in the subject. Each possibility represents a separate embodiment of the invention.

In some embodiments, the method comprises administering to the subject a therapeutically effective amount of a pharmaceutical composition comprising Flumatinib and Saroglitazar, thereby treating or preventing SARS-CoV-2 virulence in the subject. Each possibility represents a separate embodiment of the invention.

In some embodiments, the method comprises administering to the subject a synergistically effective amount of a pharmaceutical composition comprising Flumatinib and Saroglitazar, thereby treating or preventing SARS-CoV-2 virulence in the subject. Each possibility represents a separate embodiment of the invention.

In some embodiments, the method comprises administering to the subject a pharmaceutical composition comprising Flumatinib and Saroglitazar in a molar per molar ratio ranging from 100:1 to 1:100, 50:1 to 1:50, 30:1 to 1:30, 15:1 to 1:15, 10:1 to 1:10, 8:1 to 1:8, 4:1 to 1:4, 3:1 to 1:3, 2:1 to 1:2, or is 1:1, thereby treating or preventing SARS-CoV-2 virulence in the subject. Each possibility represents a separate embodiment of the invention. In some embodiments, the method comprises administering to the subject a therapeutically effective amount of a pharmaceutical composition comprising Flumatinib and Plerixafor, thereby treating or preventing SARS-CoV-2 virulence in the subject. Each possibility represents a separate embodiment of the invention.

In some embodiments, the method comprises administering to the subject a synergistically effective amount of a pharmaceutical composition comprising Flumatinib and Plerixafor, thereby treating or preventing SARS-CoV-2 virulence in the subject. Each possibility represents a separate embodiment of the invention.

In some embodiments, the method comprises administering to the subject a pharmaceutical composition comprising Flumatinib and Plerixafor in a molar per molar ratio ranging from 100:1 to 1:100, 50:1 to 1:50, 30:1 to 1:30, 15:1 to 1:15, 10:1 to 1:10, 8:1 to 1:8, 4:1 to 1:4, 3:1 to 1:3, 2:1 to 1:2, or is 1:1, thereby treating or preventing SARS-CoV-2 virulence in the subject. Each possibility represents a separate embodiment of the invention.

In some embodiments, the method comprises administering to the subject a therapeutically effective amount of a pharmaceutical composition comprising Flumatinib and Gliclazide, thereby treating or preventing SARS-CoV-2 virulence in the subject. Each possibility represents a separate embodiment of the invention.

In some embodiments, the method comprises administering to the subject a synergistically effective amount of a pharmaceutical composition comprising Flumatinib and Gliclazide, thereby treating or preventing SARS-CoV-2 virulence in the subject. Each possibility represents a separate embodiment of the invention.

In some embodiments, the method comprises administering to the subject a pharmaceutical composition comprising Flumatinib and Gliclazide in a molar per molar ratio ranging from 100:1 to 1:100, 50:1 to 1:50, 30:1 to 1:30, 15:1 to 1:15, 10:1 to 1:10, 8:1 to 1:8, 4:1 to 1:4, 3:1 to 1:3, 2:1 to 1:2, or is 1:1, thereby treating or preventing SARS-CoV-2 virulence in the subject. Each possibility represents a separate embodiment of the invention.

In some embodiments, the method comprises administering to the subject a therapeutically effective amount of a pharmaceutical composition comprising Darapladib and Mavorixafor, thereby treating or preventing SARS-CoV-2 virulence in the subject. Each possibility represents a separate embodiment of the invention.

In some embodiments, the method comprises administering to the subject a synergistically effective amount of a pharmaceutical composition comprising Darapladib and Mavorixafor, thereby treating or preventing SARS-CoV-2 virulence in the subject. Each possibility represents a separate embodiment of the invention.

In some embodiments, the method comprises administering to the subject a pharmaceutical composition comprising Darapladib and Mavorixafor in a molar per molar ratio ranging from 100:1 to 1:100, 50:1 to 1:50, 30:1 to 1:30, 15:1 to 1:15, 10:1 to 1:10, 8:1 to 1:8, 4:1 to 1:4, 3:1 to 1:3, 2:1 to 1:2, or is 1:1, thereby treating or preventing SARS-CoV-2 virulence in the subject. Each possibility represents a separate embodiment of the invention.

In some embodiments, the method comprises administering to the subject a therapeutically effective amount of a pharmaceutical composition comprising Darapladib and Cyclen, thereby treating or preventing SARS-CoV-2 virulence in the subject. Each possibility represents a separate embodiment of the invention.

In some embodiments, the method comprises administering to the subject a synergistically effective amount of a pharmaceutical composition comprising Darapladib and Cyclen, thereby treating or preventing SARS-CoV-2 virulence in the subject. Each possibility represents a separate embodiment of the invention.

In some embodiments, the method comprises administering to the subject a pharmaceutical composition comprising Darapladib and Cyclen in a molar per molar ratio ranging from 100:1 to 1:100, 50:1 to 1:50, 30:1 to 1:30, 15:1 to 1:15, 10:1 to 1:10, 8:1 to 1:8, 4:1 to 1:4, 3:1 to 1:3, 2:1 to 1:2, or is 1:1, thereby treating or preventing SARS-CoV-2 virulence in the subject. Each possibility represents a separate embodiment of the invention.

In some embodiments, the method comprises administering to the subject a therapeutically effective amount of a pharmaceutical composition comprising Darapladib and Mebrofenin, thereby treating or preventing SARS-CoV-2 virulence in the subject. Each possibility represents a separate embodiment of the invention.

In some embodiments, the method comprises administering to the subject a synergistically effective amount of a pharmaceutical composition comprising Darapladib and Mebrofenin, thereby treating or preventing SARS-CoV-2 virulence in the subject. Each possibility represents a separate embodiment of the invention.

In some embodiments, the method comprises administering to the subject a pharmaceutical composition comprising Darapladib and Mebrofenin in a molar per molar ratio ranging from 100:1 to 1:100, 50:1 to 1:50, 30:1 to 1:30, 15:1 to 1:15, 10:1 to 1:10, 8:1 to 1:8, 4:1 to 1:4, 3:1 to 1:3, 2:1 to 1:2, or is 1:1, thereby treating or preventing SARS-CoV-2 virulence in the subject. Each possibility represents a separate embodiment of the invention.

According to some embodiments, there is provided a method for treating or preventing SARS-CoV-2 virulence in a subject in need thereof, the method comprising administering to the subject a therapeutically effective amount of a pharmaceutical composition comprising Flumatinib and Darapladib.

In some embodiments, the method comprises administering to the subject a therapeutically effective amount of a pharmaceutical composition comprising Flumatinib and Darapladib in a molar per molar ratio ranging from 100:1 to 1:100, 50:1 to 1:50, 30:1 to 1:30, 15:1 to 1:15, 10:1 to 1:10, 8:1 to 1:8, 4:1 to 1:4, 3:1 to 1:3, 2:1 to 1:2, or is 1:1. Each possibility represents a separate embodiment of the invention.

According to some embodiments, there is provided a method for increasing or enhancing the anti-viral activity of Mavorixafor in a subject in need thereof, comprising administering to the subject a pharmaceutical composition comprising Flumatinib.

According to some embodiments, there is provided a method for increasing or enhancing the anti-viral activity of Cyclen in a subject in need thereof, comprising administering to the subject a pharmaceutical composition comprising Flumatinib.

According to some embodiments, there is provided a method for increasing or enhancing the anti-viral activity of Mebrofenin in a subject in need thereof, comprising administering to the subject a pharmaceutical composition comprising Flumatinib.

According to some embodiments, there is provided a method for increasing or enhancing the anti-viral activity of Saroglitazar in a subject in need thereof, comprising administering to the subject a pharmaceutical composition comprising Flumatinib.

According to some embodiments, there is provided a method for increasing or enhancing the anti-viral activity of Plerixafor in a subject in need thereof, comprising administering to the subject a pharmaceutical composition comprising Flumatinib.

According to some embodiments, there is provided a method for increasing or enhancing the anti-viral activity of Gliclazide in a subject in need thereof, comprising administering to the subject a pharmaceutical composition comprising Flumatinib.

According to some embodiments, there is provided a method for increasing or enhancing the anti-viral activity of Flumatinib in a subject in need thereof, comprising administering to the subject a pharmaceutical composition comprising Saroglitazar.

According to some embodiments, there is provided a method for increasing or enhancing the anti-viral activity of Flumatinib in a subject in need thereof, comprising administering to the subject a pharmaceutical composition comprising Plerixafor.

According to some embodiments, there is provided a method for increasing or enhancing the anti-viral activity of Flumatinib in a subject in need thereof, comprising administering to the subject a pharmaceutical composition comprising Gliclazide.

According to some embodiments, there is provided a method for increasing or enhancing the anti-viral activity of Flumatinib in a subject in need thereof, comprising administering to the subject a pharmaceutical composition comprising Mavorixafor.

According to some embodiments, there is provided a method for increasing or enhancing the anti-viral activity of Flumatinib in a subject in need thereof, comprising administering to the subject a pharmaceutical composition comprising Cyclen.

According to some embodiments, there is provided a method for increasing or enhancing the anti-viral activity of Flumatinib in a subject in need thereof, comprising administering to the subject a pharmaceutical composition comprising Mebrofenin.

According to some embodiments, there is provided a method for increasing or enhancing the anti-viral activity of Mavorixafor in a subject in need thereof, comprising administering to the subject a pharmaceutical composition comprising Darapladib.

According to some embodiments, there is provided a method for increasing or enhancing the anti-viral activity of Cyclen in a subject in need thereof, comprising administering to the subject a pharmaceutical composition comprising Darapladib.

According to some embodiments, there is provided a method for increasing or enhancing the anti-viral activity of Mebrofenin in a subject in need thereof, comprising administering to the subject a pharmaceutical composition comprising Darapladib.

According to some embodiments, there is provided a method for increasing or enhancing the anti-viral activity of Darapladib in a subject in need thereof, comprising administering to the subject a pharmaceutical composition comprising Mavorixafor.

According to some embodiments, there is provided a method for increasing or enhancing the anti-viral activity of Darapladib in a subject in need thereof, comprising administering to the subject a pharmaceutical composition comprising Cyclen.

According to some embodiments, there is provided a method for increasing or enhancing the anti-viral activity of Darapladib in a subject in need thereof, comprising administering to the subject a pharmaceutical composition comprising Mebrofenin.

According to another aspect, there is provided a method for increasing or enhancing the anti-viral activity of Flumatinib in a subject in need thereof, comprising administering to the subject a pharmaceutical composition comprising Darapladib.

According to another aspect, there is provided a method for increasing or enhancing the anti-viral activity of Darapladib in a subject in need thereof, comprising administering to the subject a pharmaceutical composition comprising Flumatinib.

In some embodiments, a therapeutically effective dose of the composition of the invention is administered. The term “therapeutically effective amount” refers to an amount of a drug effective to treat a disease or disorder in a mammal. The term “a therapeutically effective amount” refers to an amount effective, at dosages and for periods of time necessary, to achieve the desired therapeutic or prophylactic result. The exact dosage form and regimen would be determined by the physician according to the patient's condition.

The dosage administered will be dependent upon the age, health, and weight of the recipient, kind of concurrent treatment, if any, frequency of treatment, and the nature of the effect desired. The route of administration of the pharmaceutical compositions will depend on the disease or condition to be treated. Suitable routes of administration include, but are not limited to, parenteral injections, e.g., intradermal, intravenous, intramuscular, intralesional, subcutaneous, intrathecal, and any other mode of injection as known in the art. Although the bioavailability of peptides administered by other routes can be lower than when administered via parenteral injection, by using appropriate compositions it is envisaged that it will be possible to administer the compositions of the invention via transdermal, oral, rectal, vaginal, topical, nasal, inhalation and ocular modes of treatment. In addition, it may be desirable to introduce the pharmaceutical compositions of the invention by any suitable route, including intraventricular and intrathecal injection; intraventricular injection may be facilitated by an intraventricular catheter, for example, attached to a reservoir.

In some embodiments, the composition of the invention is delivered orally. In some embodiments, the composition of the invention is an oral composition. In some embodiments, the composition of the invention further comprises orally acceptable carrier, excipient, or a diluent.

According to some embodiments, the active agents of the invention (e.g., SARS-CoV-2 E protein channel blocker or protein 3a inhibiter) is for use at a daily dose of 0.01 to 500 mg/kg.

According to some embodiments, the SARS-CoV-2 E protein channel blocker is Memantine or a salt thereof and is for use at a daily dose of between about 1 mg/day and about 50 mg/day, about 1 mg/day and 45 mg/day, and 5 mg/day and 3 5 mg/day.

According to some embodiments, the SARS-CoV-2 E protein channel blocker is Gliclazide or a salt thereof and is for use at a daily dose of between about 1 mg/day and 350 mg/day, 10 mg/day and 350 mg/day, 50 mg/day and 350 mg/day, 1 mg/day and 300 mg/day, 10 mg/day and 300 mg/day, and 50 mg/day and 250 mg/day.

According to some embodiments, the SARS-CoV-2 E protein channel blocker is Mavorixafor or a salt thereof and is for use at a daily dose of between about 50 mg/day and about 100 mg/day, about 50 mg/day and 200 mg/day, and 50 mg/day and 400 mg/day.

According to some embodiments, the SARS-CoV-2 E protein channel blocker is Saroglitazar or a salt thereof and is for use at a daily dose of between about 0.1 mg/day and about 5 mg/day, about 1 mg/day and 4 mg/day, and 1.5 mg/day and 4.5 mg/day.

According to some embodiments, the SARS-CoV-2 E protein channel blocker is Mebrofenin or a salt thereof and is for use at a daily dose of between about 1 mg/day and about 50 mg/day, about 1 mg/day and 45 mg/day, and 5 mg/day and 35 mg/day.

According to some embodiments, the SARS-CoV-2 E protein channel blocker is Cyclen or a salt thereof and is for use at a daily dose of between about 0.01 mg/day and about 0.5 mg/day, about 0.1 mg/day and 0.5 mg/day, and 0.05 mg/day and 0.3 mg/day.

According to some embodiments, the SARS-CoV-2 E protein channel blocker, the SARS-CoV-2 3a protein inhibitor, or both is Kasugamycin or a salt thereof and is for use at a daily dose of between about 1 mg/day and about 500 mg/day, about 5 mg/day and 250 mg/day, and 10 mg/day and 350 mg/day.

According to some embodiments, the SARS-CoV-2 E protein channel blocker is Azacytidine or a salt thereof and is for use at a daily dose of between about 1 mg/day and about 100 mg/day, about 1 mg/day and 200 mg/day, and 1 mg/day and 300 mg/day.

According to some embodiments, the SARS-CoV-2 E protein channel blocker, the SARS-CoV-2 3a protein inhibitor, or both is Plerixafor or a salt thereof and is for use at a daily dose of between about 1 mg/day and about 50 mg/day, about 1 mg/day and 45 mg/day, and 5 mg/day and 35 mg/day.

According to some embodiments, the SARS-CoV-2 3a protein inhibitor is Capreomycin or a salt thereof and is for use at a daily dose of between about 50 mg/day and about 1,000 mg/day, about 10 mg/day and 700 mg/day, and 20 mg/day and 800 mg/day.

According to some embodiments, the SARS-CoV-2 3a protein inhibitor is Pentamidine or a salt thereof and is for use at a daily dose of between about 50 mg/day and about 500 mg/day, about 30 mg/day and 400 mg/day, and 100 mg/day and 300 mg/day.

According to some embodiments, the SARS-CoV-2 3a protein inhibitor is Spectinomycin or a salt thereof and is for use at a daily dose of between about 500 mg/day and about 5,000 mg/day, about 250 mg/day and 2,500 mg/day, and 100 mg/day and 4,500 mg/day.

According to some embodiments, the SARS-CoV-2 3a protein inhibitor is Flumatinib or a salt thereof and is for use at a daily dose of between about 50 mg/day and about 1,000 mg/day, about 100 mg/day and 1,500 mg/day, and 50 mg/day and 5,000 mg/day.

According to some embodiments, the SARS-CoV-2 3a protein inhibitor is Litronesib or a salt thereof and is for use at a daily dose of between about 10 mg/day and about 3,000 mg/day, about 50 mg/day and 2,500 mg/day, and 20 mg/day and 2,000 mg/day.

According to some embodiments, the SARS-CoV-2 3a protein inhibitor is Darapladib or a salt thereof and is for use at a daily dose of between about 10 mg/day and about 1,000 mg/day, about 50 mg/day and 500 mg/day, and 100 mg/day and 800 mg/day.

According to some embodiments, the SARS-CoV-2 3a protein inhibitor is Floxuridine or a salt thereof and is for use at a daily dose of between about 1 mg/day and about 100 mg/day, about 5 mg/day and 80 mg/day, and 10 mg/day and 100 mg/day.

According to some embodiments, the SARS-CoV-2 3a protein inhibitor is Fludarabine or a salt thereof and is for use at a daily dose of between about 1 mg/day and about 100 mg/day, about 2 mg/day and 80 mg/day, and 5 mg/day and 60 mg/day.

In some embodiments, the pharmaceutical composition comprises a pharmaceutically acceptable carrier, adjuvant or excipient.

As used herein, the term “carrier,” “adjuvant” or “excipient” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelles, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

### Screening Assays

According to some embodiments, there is provided a method of screening effectiveness of an agent in treating or preventing a coronavirus infection, the method comprising providing a cell comprising a membrane permeabilized coronavirus E or 3a protein, contacting the cell with the agent, and determining effect of the agent on growth of the cell, wherein a substantial effect of the agent on cellular growth is indicative of the agent as being effective for treating or preventing a coronavirus infection, thereby screening effectiveness of an agent in treating or preventing a coronavirus infection.

In some embodiments, the method is a negative assay. In some embodiments, the cell is characterized by growth retardation due to the membrane permeabilized E protein or 3a protein. In some embodiments, an agent that alleviates growth retardation is indicative as being effective for treating or preventing a coronavirus infection.

In some embodiments, the method is a positive assay. In some embodiments, the cell is a K⁺-uptake deficient cell grown in low [K⁺] media experience growth, due to the channel formed by the E protein or 3a protein. In some embodiments, an agent that induces growth retardation is indicative as being effective for treating or preventing a coronavirus infection.

In some embodiments, the coronavirus is SARS-CoV. In some embodiments, SARS-CoV is any one of SARS-CoV-1 and SARS-CoV-2. In some embodiments, the coronavirus E protein or 3a protein is a SARS-CoV-1 E protein or 3a protein, respectively. In some embodiments, the coronavirus E protein or 3a protein is a SARS-CoV-2 E protein, or 3a protein, respectively.

In some embodiments, the method comprises performing both the negative assay and the positive assay.

In some embodiments, the cell is a bacterial cell. In some embodiments, the cell is devoid of endogenous potassium uptake, besides an exogenously provided (e.g., expressed) membrane permeabilized SARS-CoV E protein or by the 3a protein.

Non-limiting examples for growing a bacterial cell applicable for the screening methods provided herein, include: Astrahan, P. et al., Acta 1808, 394-8 (2011); Santner, P. et al. Biochemistry 57, 5949-5956 (2018), and Taube, R., Alhadeff, R., Assa, D., Krugliak, M. & Arkin, I. T. PLoS One 9, e105387 (2014).

In some embodiment, the assay is for determining susceptibility of the virus to develop resistance against the agent.

As used herein, the term “about” when combined with a value refers to plus and minus 10% of the reference value. For example, a length of about 1,000 nanometers (nm) refers to a length of 1000 nm±100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein, and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Strategies for Protein Purification and Characterization—A Laboratory Course Manual” CSHL Press (1996); all of which are incorporated by reference. Other general references are provided throughout this document.

### Example 1

### Materials and Methods

### Bacterial Strains

Three strains of K12 Escherichia coli were used in the current study: DH10B, LB650, and LR1. DH10B cells were purchased from Invitrogen (Carlsbad, Calif.). LB650 bacteria (ΔtrkG, ΔtrkH, and ΔkdpABC5 system) contain deletions in genes connected to potassium uptake (Stumpe, S. & Bakker, E. P. Arch Microbiol 167, 126-36 (1997)). LR1 bacteria contained a chromosomal copy of a pH sensitive green fluorescence protein (GFP) called pHluorin (Miesenbock, G and De Angelis, D A and Rothman, J E. Nature 394, 192-5 (1998)).

### Plasmids

The SARS-CoV-2 E protein, 3a protein, and the influenza M2 channel were expressed as fusion proteins to the maltose binding protein using the pMAL-p2X plasmid (New England Biolabs, Ipswich, Mass.). Genes for the viral proteins have been added with a nucleotide sequence coding for linker of seven amino-acids, six histidines, and a stop codon at the 3′ end. EcoRI and XbaI restriction sites were located at the 5′ and 3′ ends, respectively. The sequences were synthesized by GenScript (Piscataway, N.J.). Protein expression was achieved by adding isopropyl β-D-1-thiogalactopyranoside (IPTG) to the growth media, as indicated.

### Chemicals

IPTG was purchased from Biochemika-Fluka (Buchs, Switzerland). All other chemicals were purchased from Sigma-Aldrich laboratories (Rehovot, Israel).

### Growth Media

Lysogeny Broth (LB) was used for all bacterial growth (Bertani, G. J Bacteriol 62, 293-300 (1951)) unless noted otherwise. LBK was similar to LB expect that KCl replaces NaCl at 10 gr/L. All media contained ampicillin at 50 μg/ml.

### Bacterial Growth

Escherichia coli DH10B bacteria bearing or lacking (as a reference) the viral chimera were grown overnight in LB at 37° C. Thereafter, the growth culture was diluted and the bacteria were grown until their O.D.₆₀₀ reached 0.2. Fifty (50) μl of bacterial culture were subsequently dispensed into 96-well flat-bottomed plates (Nunc, Roskilde, Denmark) containing 50 μl of the different treatments. Unless stated otherwise, IPTG was added to the cells to final concentrations ranging from 0 to 100 μM. D-glucose was added to a concentration of 1%. Ninety six (96)-well plates were incubated for 16 hours at 37° C. in an Infinite 200 from the Tecan Group (Männedorf, Switzerland) at a constant high shaking rate. O.D.₆₀₀ readings were recorded every 15 min. For every measurement duplicates or triplicates were conducted.

For the Escherichia coli LB650 bacteria, the same protocol was used, except that growth was done in LBK overnight. Thereafter, the growth medium was replaced with LB and the bacteria were diluted and grown until their O.D.₆₀₀ reached 0.2, and diluted twofold with the various treatments in each well. Unless stated otherwise IPTG was added to the LB650 bacteria to a final concentration of 10 μM.

pHlux Assay

Transformed LR1 cells were cultured overnight in LB media containing 1% glucose and 50 μM ampicillin. Secondary cultures were prepared by diluting the primary culture by 1:500 in LB media and allowing it to grow to an O.D.₆₀₀ of 0.6-0.8. Protein synthesis was induced by the addition of 50 μM IPTG for two hours. Cultures without IPTG induction were used as control. Following two hours of induction, the O.D.₆₀₀ of all cells were measured, and after pelleting at 3,500 g for 10 min, the bacteria were resuspended in McIlvaine buffer (200 mM Na₂HPO₄, 0.9% NaCl adjusted to pH 7.6 with 0.1 M citric acid, 0.9% Nacl) to an optical density of 0.25 at 600 nm. 200 μl of cell suspension were subsequently transferred with 30 μl of McIlvaine buffer to 96 well plate. The plate includes a row with only assay buffer and cultures without induction. The fluorescence measurements were carried out in a microplate reader (Infinite F200 pro, Tecan) with the emission set at 520 nm, while excitation shifted between 390 nm and 466 nm.

A liquid handling system (Tecan) was used to add 70 μl of 300 mM citric acid with 0.9% NaCl to the bacteria. The fluorescence emission of each well after addition of acid was measured by alternate read out of the two filter pairs for 50 seconds. The ratio for the two differently excited emissions, F=F_{390 nm}/F_{466 nm} was calculated and translated into proton concentration using the following equation:

[H⁺]=0.132·F^{-1.75·F}^{^{0.51}}

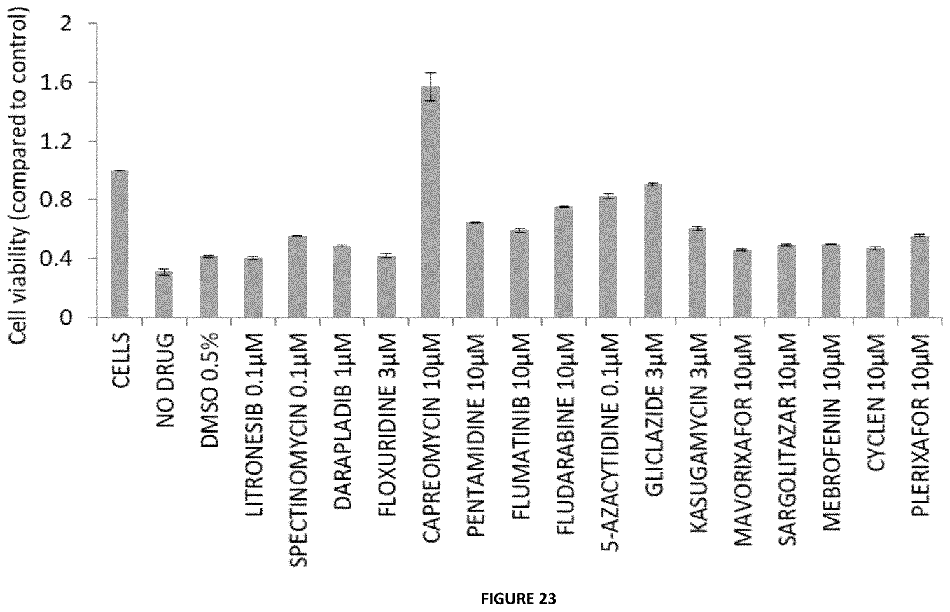

Vero-E6 cells were pretreated for 20 h with tested compounds (drugs including their concentration are described in FIG. 23) and were infected with SARS-CoV-2 at a multiplicity of infection (MOI) of 0.001 and 0.01 in the presence of the compounds as indicated. The medium with the same DMSO concentration was used as the no-drug control. Drug efficacies in control of toxicity were assessed at 24 hours post infection by MTT assay. All infection experiments were performed in a BSL-3 facility.

### Results

Three, recently developed bacteria-based assays (Astrahan, P. et al. Biochim Biophys Acta 1808, 394-8 (2011); Santner, P. et al. Biochemistry 57, 5949-5956 (2018); Taube, R., Alhadeff, R., Assa, D., Krugliak, M. & Arkin, I. T. PLoS One 9, e105387 (2014); Tomar, P. P. S., Oren, R., Krugliak, M. & Arkin, I. T. Viruses 11 (2019)) were tailord to examine if SARS-CoV-2 E protein is an ion channel.

These assays are quantitative, easy to implement rapidly, and are amenable to high-throughput screening for inhibitor identification. Moreover, each of these there assays was used on known viroporins and were shown to distinguish non-conducting transmembrane domains (Tomar, P. P. S., Oren, R., Krugliak, M. & Arkin, I. T., Viruses 11 (2019)). Finally, one of the assays can also be used to predict, prior to clinical use, the options that the virus has to develop resistance against any particular inhibitor of the channel (Assa, D., Alhadeff, R., Krugliak, M. & Arkin, I. T., J. Mol Biol 428, 4209-4217 (2016)).

In order to ensure proper membrane incorporation, the inventors made use of of the pMAL protein fusion and purification system (New England Biolabs). In this construct which has been used successfully with multiple viroporins, SARS-CoV-2 E protein or 3a protein are fused to the carboxyl terminus of the periplasmic maltose binding protein. As a positive control, the inventors compared the activity of the proteins to the M2 channel from the influenza A virus, the archetypical viroporin that can be inhibited by aminoadamantanes (Pinto, L. H., Holsinger, L. J. & Lamb, R. A. Cell 69, 517-28 (1992)).

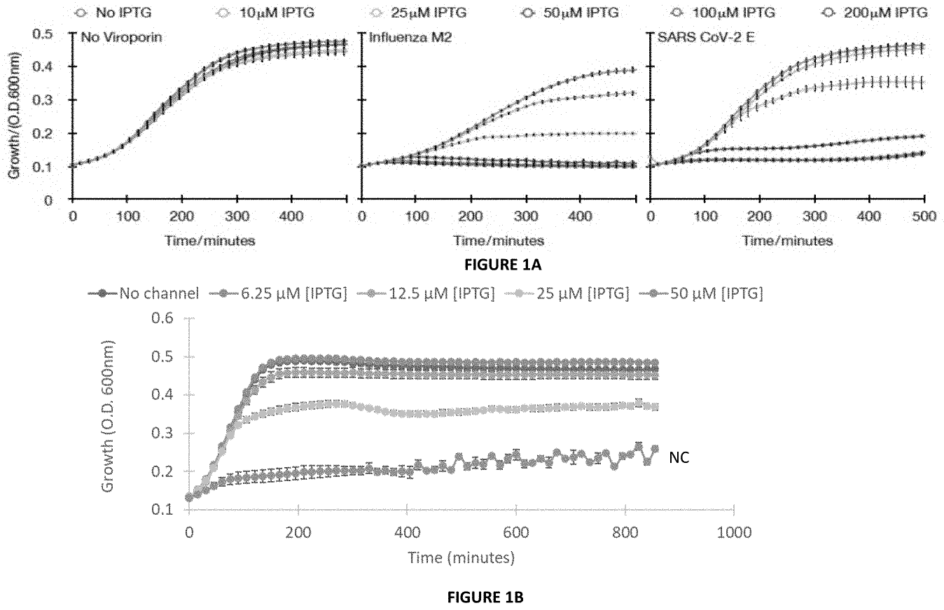

The first test that was undertaken was to examine if the SARS-CoV-2 E and 3a proteins' channel activity can lead to membrane permeabilization and thereby negatively impact bacterial growth (negative genetic test). As seen in numerous other viroporins, when expressed at increasing levels, channel activity hampers growth due to its deleterious impact on the proton motive force. Subsequently, channel-blocking drugs may be identified readily due to their ability to alleviate growth retardation. The data in FIG. 1 show that expression of the SARS-CoV-2 E or 3a protein causes significant bacterial growth retardation proportional to the protein's expression levels. This behaviour is similar to that of a known proton channel, the M2 influenza A protein.

The inventors recognized that growth retardation is not an uncommon consequence of heterologous protein expression in bacteria. In other words, spurious factors could lead to bacterial death in addition to channel activity. The inventors thus demonstrate that bacterial death is due to protein channel activity in the following three ways: (i) The inventors identify E and 3a protein channel blockers and show that they can revive bacterial growth; (ii) The inventors developed a complementary bacterial growth assay, where channel activity is essential for growth (positive genetic test); and (iii) The inventors show that protein expression increases H⁺ conductivity in an assay involving a pH sensitive GFP (Santner, P. et al. Biochemistry 57, 5949-5956 (2018)).

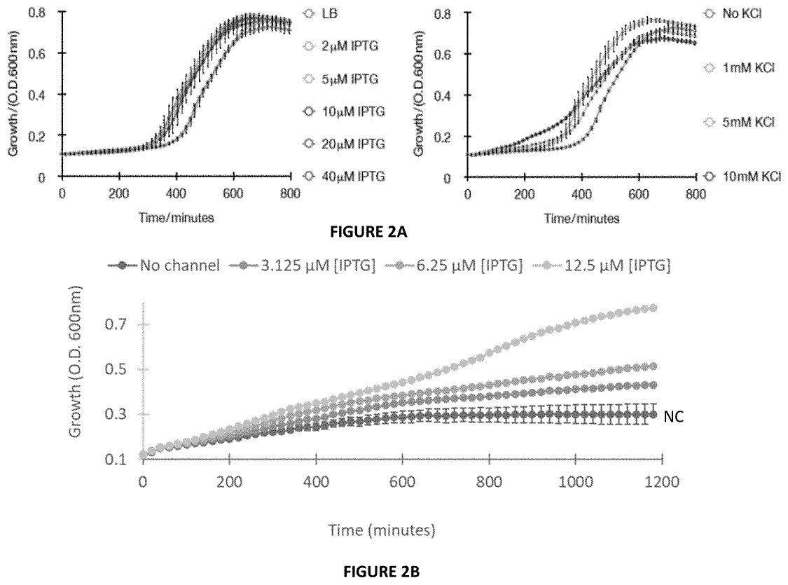

The second experimental test that the inventors have performed examines K⁺ conductivity. Specifically, K⁺-uptake deficient bacteria (Stumpe, S. & Bakker, E. P. Arch Microbiol 167, 126-36 (1997)) are incapable of growth, unless the media is supplemented by K⁺. However, when a channel capable of K⁺ transport is heterologously expressed, the bacteria can thrive even under low K⁺ media (Taube, R. et al. PLoS One 9, e105387 (2014); Tomar, P. P. S., Oren, R., Krugliak, M. & Arkin, I. T. Viruses 11 (2019)). Hence, in this instance the viral channel is essential to bacterial growth (positive genetic test). Finally, results shown in FIG. 2 indicate that expression of SARS-CoV-2 E or 3a proteins are able to increase the growth rate of K⁺-uptake deficient bacteria in otherwise limiting conditions (i.e., low [K⁺]).

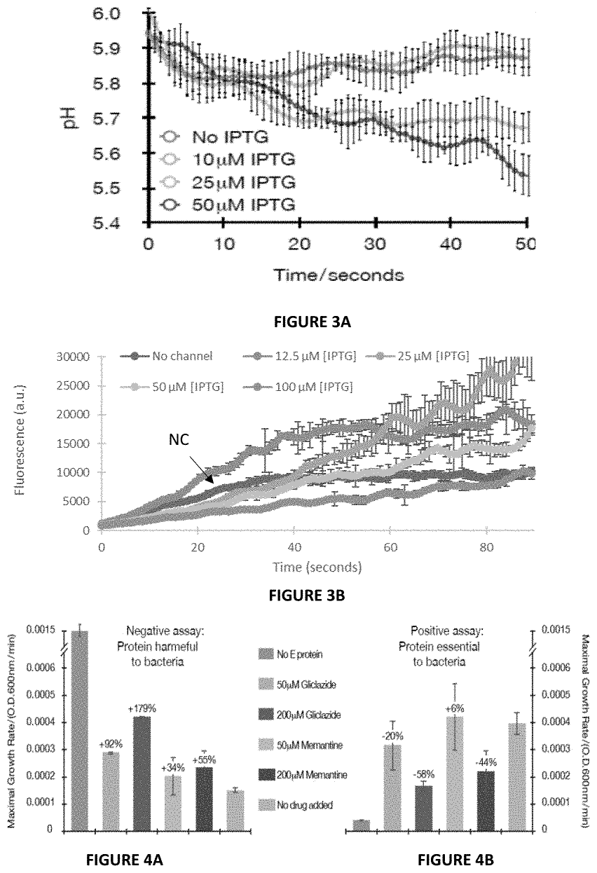

The final test to examine channel activity, was based on detecting protein-mediated H⁺ flux in bacteria that express a pH-sensitive green fluorescent protein (Santner, P. et al. Biochemistry 57, 5949-5956 (2018)). The addition of an acidic solution to the media will result in cytoplasmic acidification if the bacteria express a channel capable of H⁺ transport. Consequently, as seen in FIG. 3, expression of the E or 3a proteins from SARS-CoV-2 results in appreciable cytoplasmic acidification, indicative of its ability to transport protons. Similar acidification was detected in other viroporins, such as the influenza A M2 channel.

Considering that all three bacterial assays indicated that the SARS-CoV-2 E and 3a proteins are a potential viroporins, the inventors set forth to screen a small data set of known channel blockers. First, a library of 372 compounds from MedChemExpress (NJ, USA) in the area of “Membrane Transporter/Ion Channel”. Each of these chemicals was tested in the positive and negative genetic tests detailed above against the E protein.

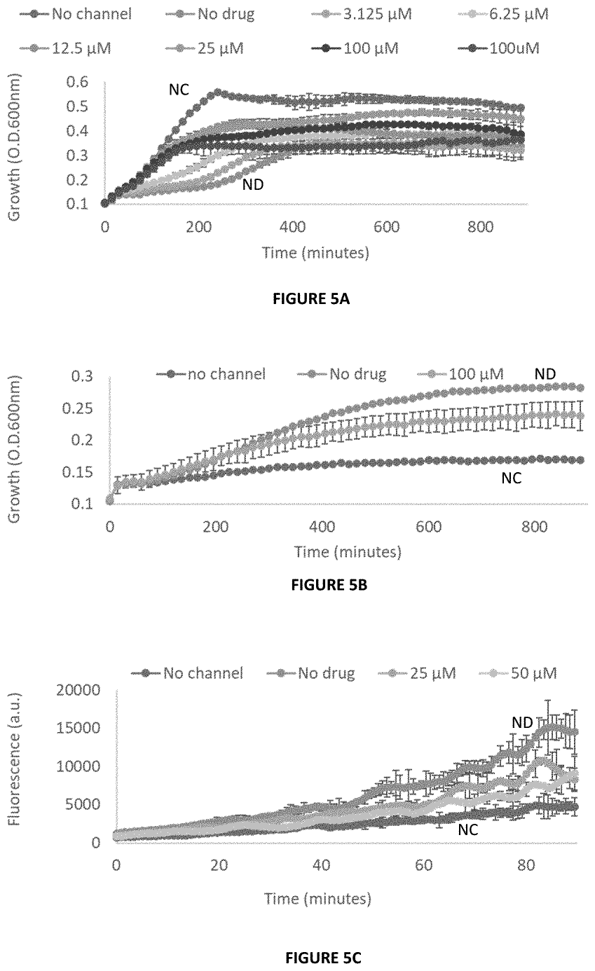

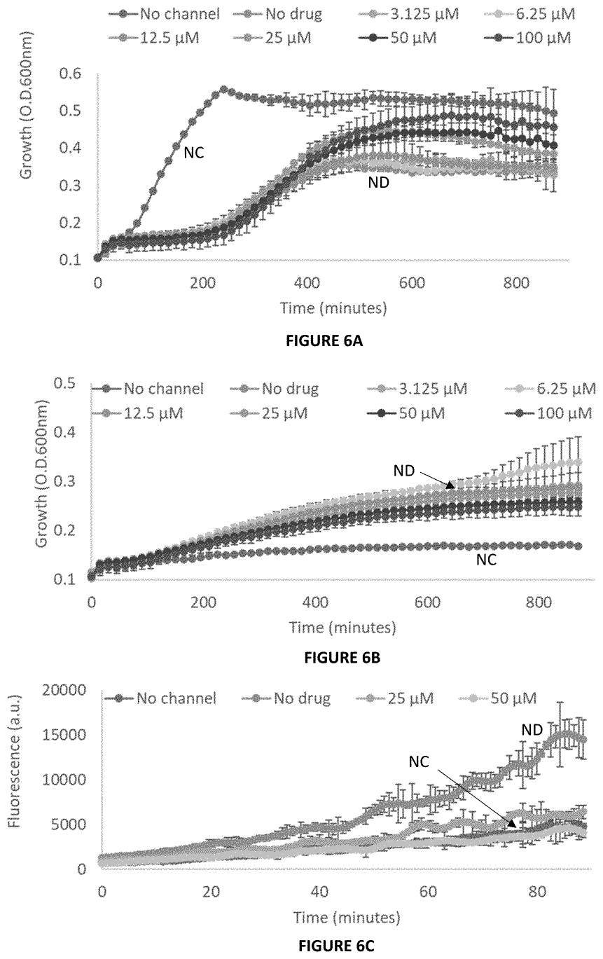

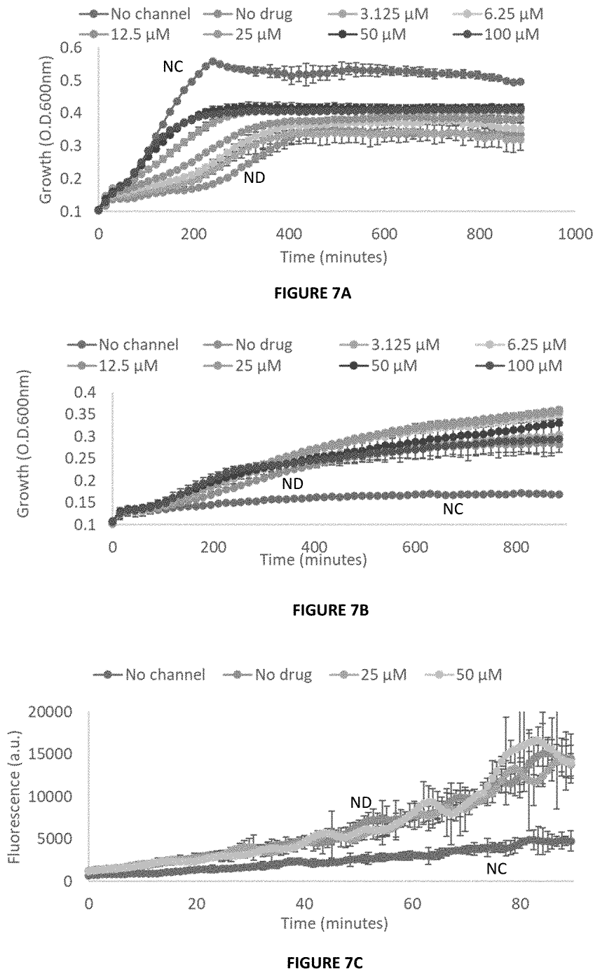

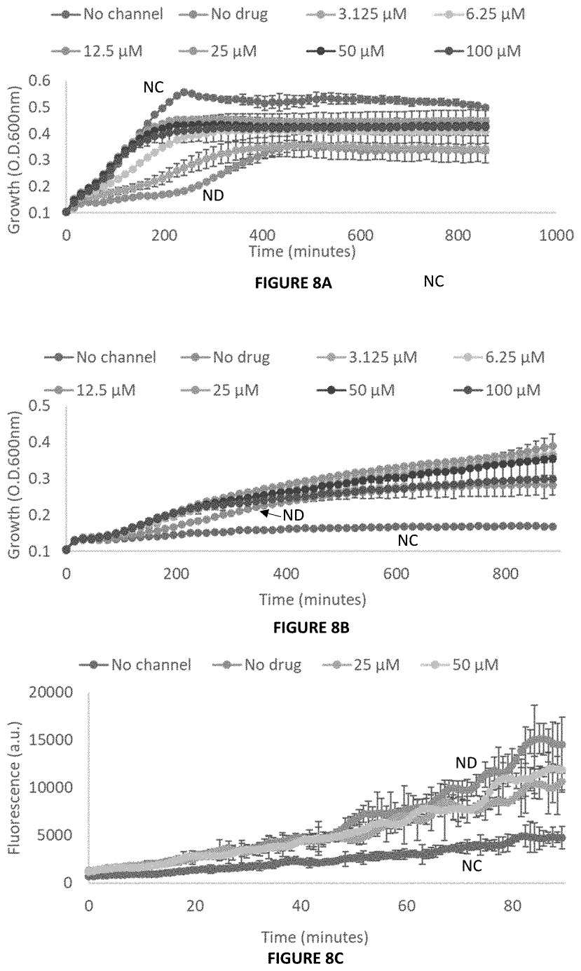

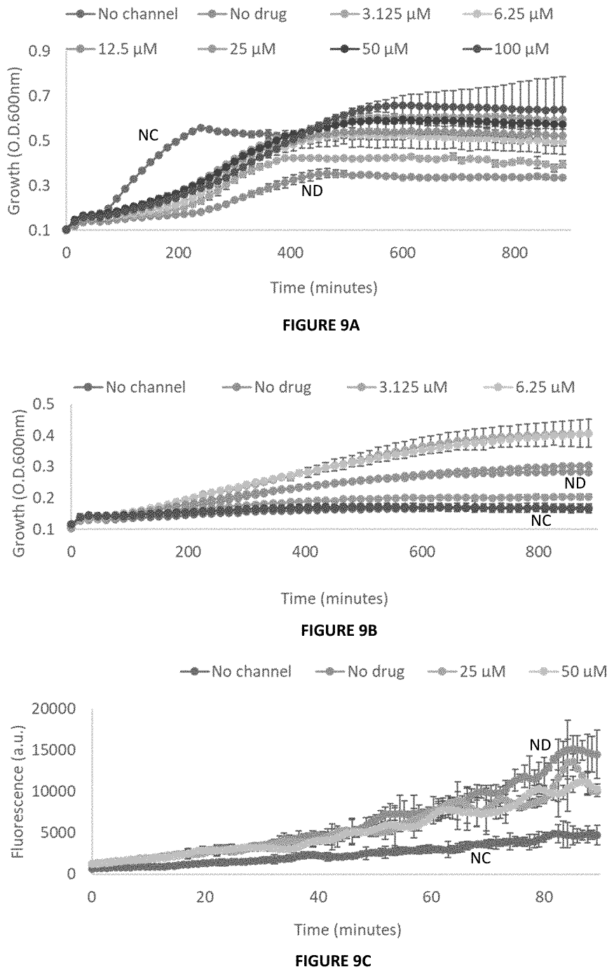

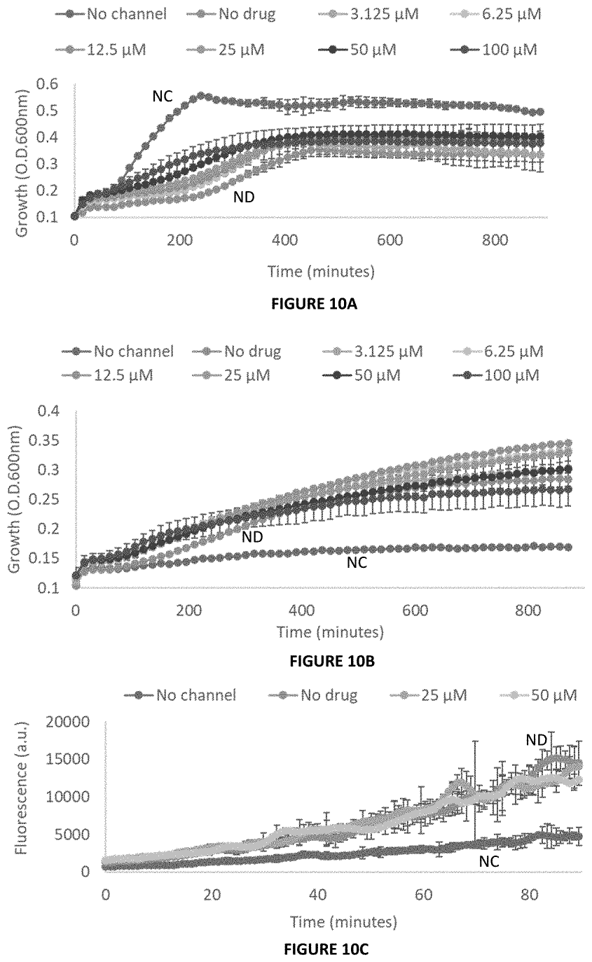

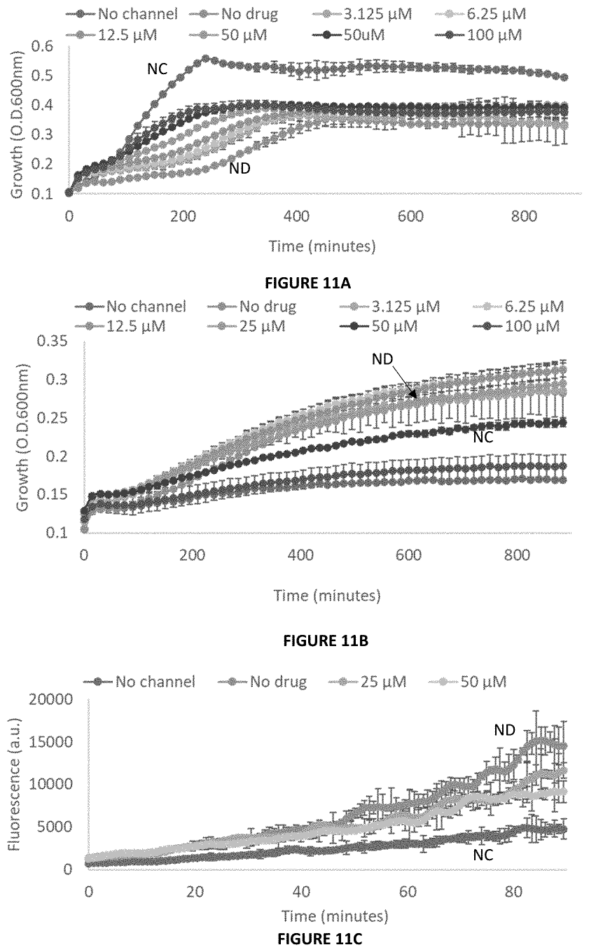

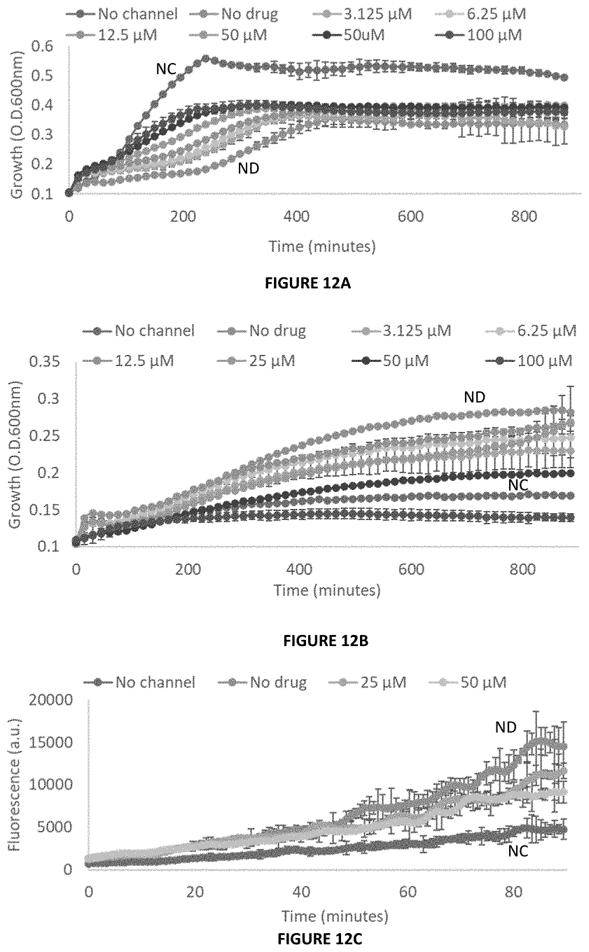

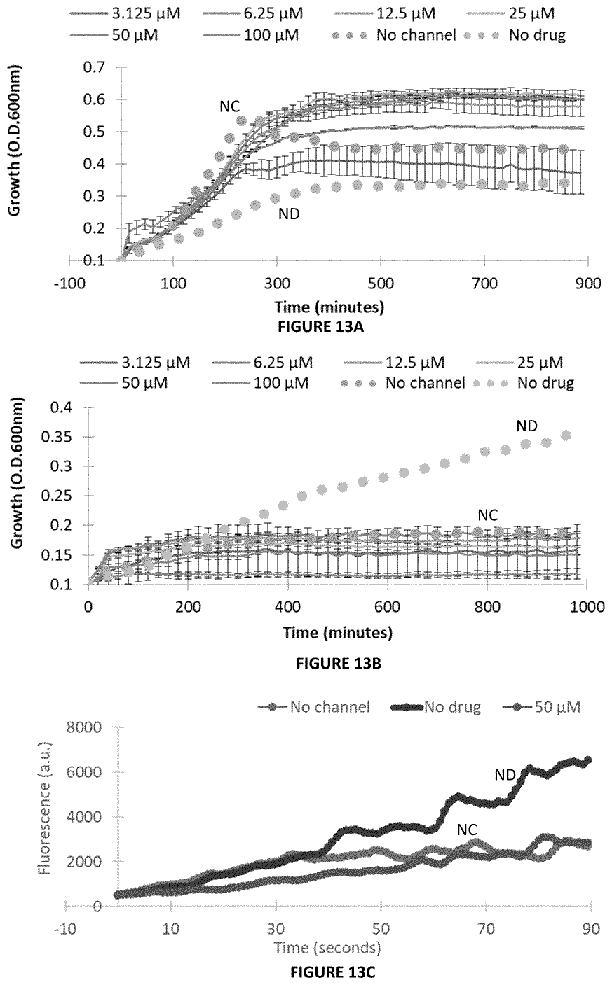

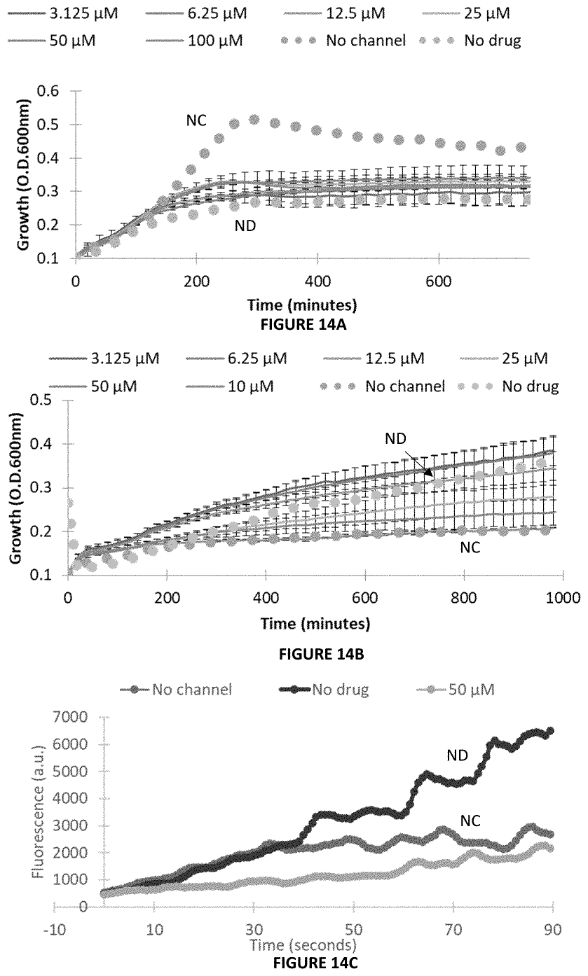

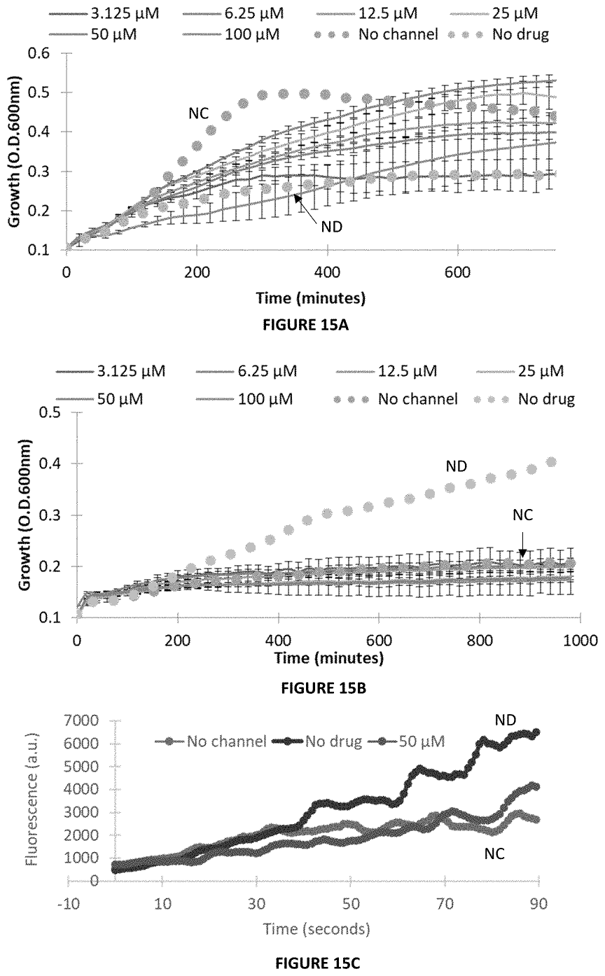

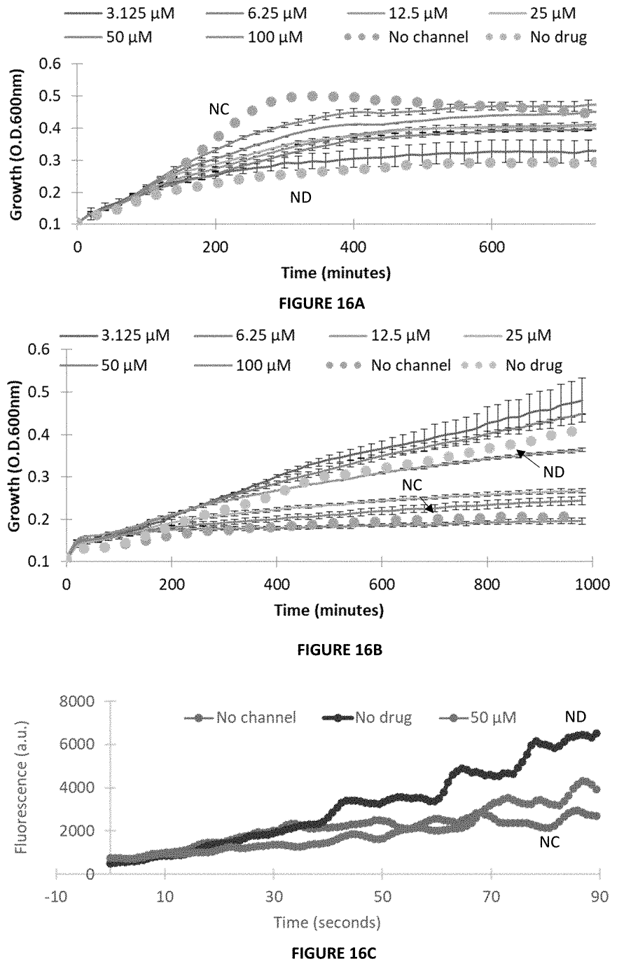

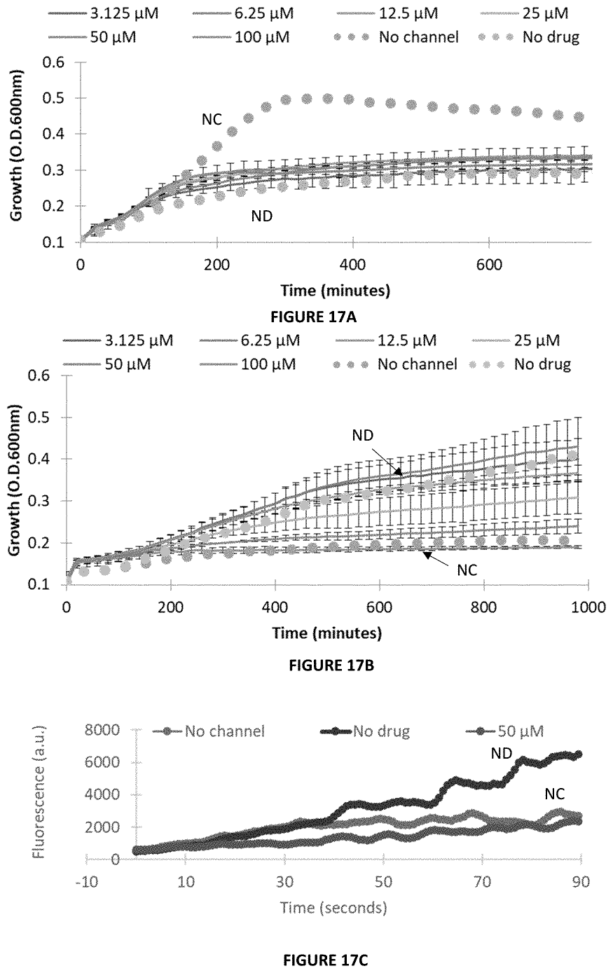

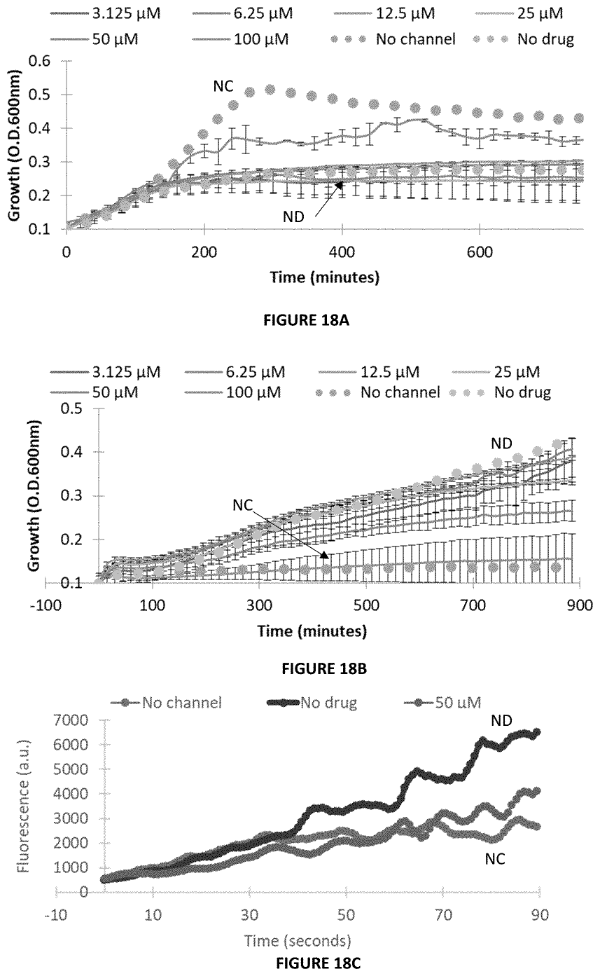

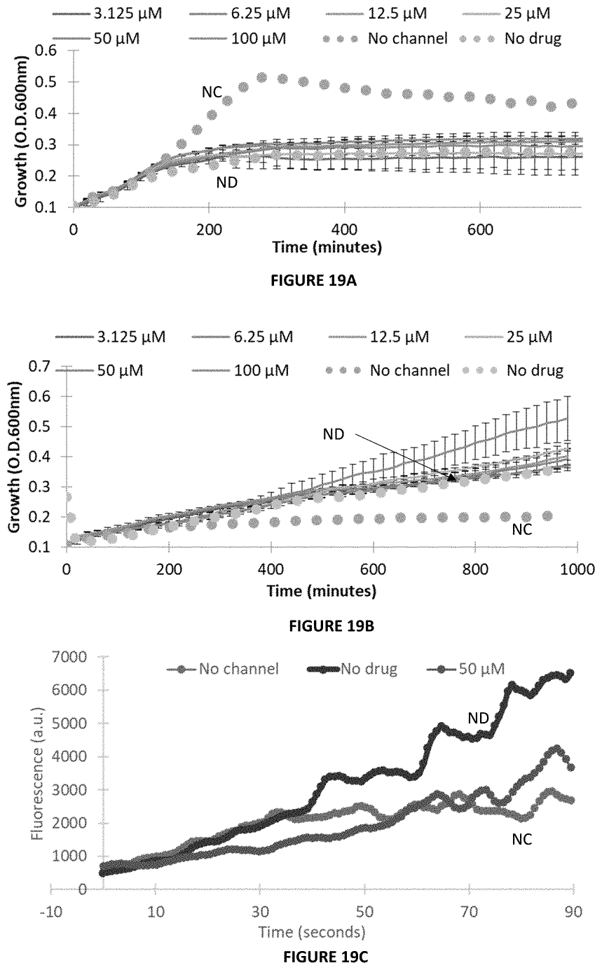

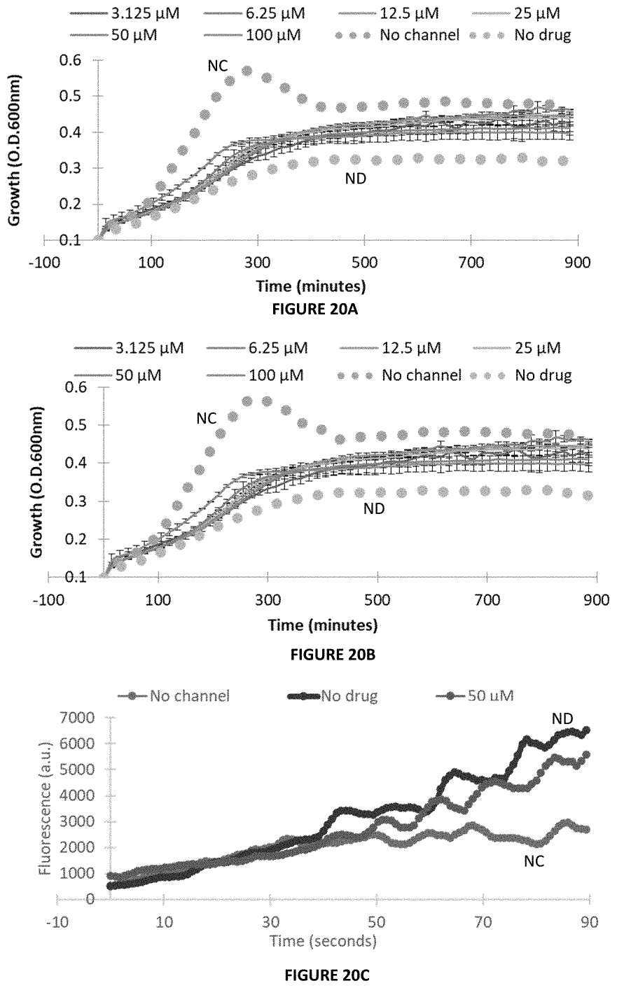

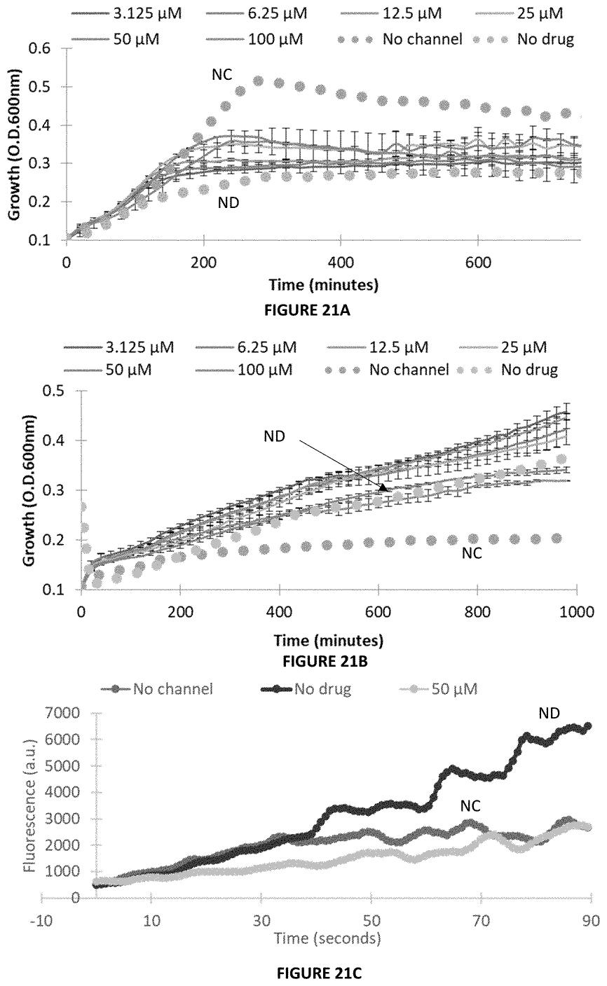

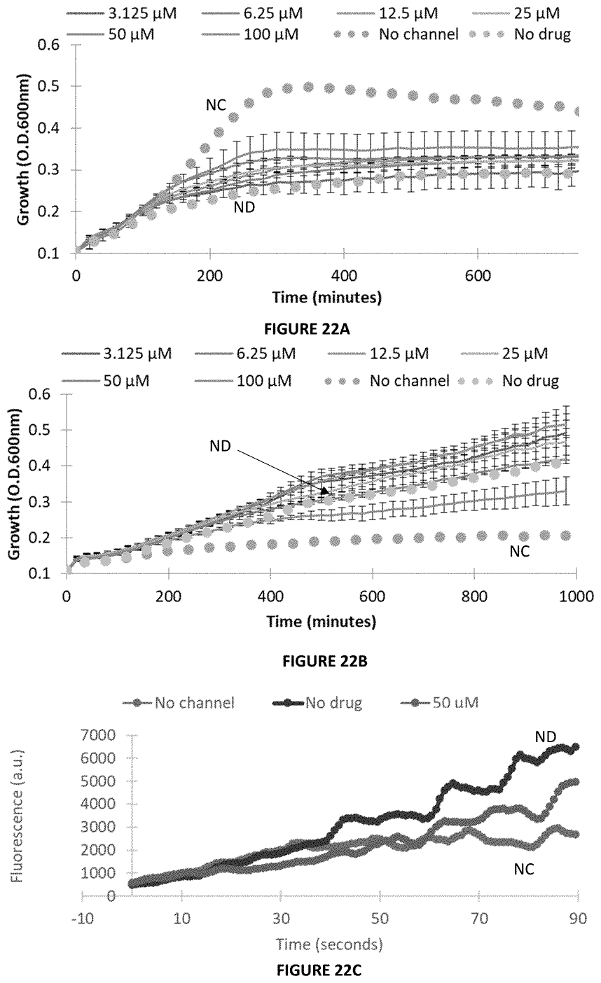

In the negative assay, bacteria experience appreciable growth retardation due to the expression of the SARS-CoV-2 E protein at elevated levels (FIG. 1). Therefore, channel blockers can be readily identified since they alleviate this growth retardation. Note that this screen inherently reduces potential toxicity since it selects for chemicals that are not toxic to the bacteria. Specifically, each of the chemicals in the pilot library was added to the media, followed by growth recording and comparison to bacteria that did not receive any treatment. Out of the 372 compound drug library, several chemicals relieved the growth inhibition that the bacteria experienced due to the SARS-CoV-2 E protein activity. Of particular notice are Gliclazide and Memantine that enhance bacterial growth, as shown in FIG. 4A.

In the positive assay screening, a reciprocal picture is obtained. K⁺-uptake deficient bacteria grown in low [K⁺] media experience growth enhancement due to the (low level) expression of the SARS-CoV-2 E protein (FIG. 2). Therefore, channel blockers can be identified since they result in growth retardation. In a manner similar to the negative assay, each of the chemicals in the pilot library was added to the media followed by growth recording. Once again, Gliclazide and Memantine scored positively in the test, in that they both inhibited growth (FIG. 4B).

The above results are encouraging since the same chemicals scored positively in reciprocal assays. Scoring positively in both assays rules out any spurious factors. When the E protein is detrimental to bacteria, the chemicals enhanced growth. However, when the E protein is essential to bacteria, the same compounds were deleterious to growth.

Our results demonstrate that the SARS-CoV-2 E protein is an ion channel. Since coronavirus E proteins are essential to virulence, it represents an attractive drug target. Our screening efforts identified two inhibitors that block E protein channel activity. Since both drugs are approved for human use for other indications, they represent candidates for swift mitigation the COVID-19 crisis.

Further, the screen was broadened and 3000 molecules were screened against the SARS-CoV-2 E protein, and 3,000 molecules were screened against SARS-CoV-2 3a protein.

The results indicate that any one of: Memantine, Gliclazide, Mavorixafor, Saroglitazar Magnesium, Mebrofenin, Cyclen, Kasugamycin, Azacytidine, and Plerixafor can be used as SARS-CoV-2 E protein channel blocker, and accordingly be used as attractive COVID-19 drugs.

The results further indicate that any one of Capreomycin, Pentamidine, Spectinomycin, Kasugamycin, Plerixafor, Flumatinib, Litronesib, Darapladib, Floxuridine, and Fludarabine, can be used as SARS-CoV-2 3a protein inhibitor, and accordingly be used as attractive COVID-19 drugs.

Further, the inventors showed the effect of the compounds tested herein on the viability of cells infected with SARS-CoV-2 virus. Specifically, the results show there was ˜60% reduction in the cell viability of the Vero-E6 cells when infected with SARS-CoV-2 at a multiplicity of infection (MOI) of 0.01, whereas the cells pretreated with the drugs showed ˜10-50% reduction of the cell viability after infected with the virus (FIG. 23).

### Example 2

### Materials and Methods

Growth of Vero E6 cells in the presence of SARS-CoV-2 were examined. Cells were grown in 96-well plates with a culture density of 10,000 cells per well. The culture media contained 10% FCS. After 24 hours, the culture media was replaced to media with 2% FCS and appropriate drugs with a final DMSO concentration of 0.1%. After one hour the cells were infected with virus at an multiplicity of infection (MOI) of 0.01. After 48 hours cell viability was monitored by MTS according to standard protocol from the manufacturer (Promega, USA).

Results were normalized relative to uninfected cells. Further comparisons may be gained by comparing the data to untreated cells and to cells that received only 0.1% DMSO. All infection experiments were performed in a BSL-3 facility.

### Screen in Bacteria

Two channels were targeted for inhibition: 3a and E.

Using three bacteria-based assays, the inventors screened a library of 2,839 approved for human use drugs and identified ten inhibitors against 3a and eight against E (Table 1).

The screen was at high concentration (100 μM)

TABLE 1
E protein and 3a protein inhibitors

| E inhibitors | 3a inhibitors |
|---|---|
| Mavorixafor (trihydrochloride) | Capreomycin (sulfate) |
| Saroglitazar Magnesium | Pentamidine (isethionate) |
| Mebrofenin | Spectinomycin (dihydrochloride) |
| Cyclen (norgestimate) | Kasugamycin (hydrochloride |
| | hydrate) |
| Kasugamycin (hydrochloride | Plerixafor |
| hydrate) | |
| 5-Azacytidine | Flumatinib |
| Plerixafor (octahydrochloride) | Litronesib (LY2523355) |
| Plerixafor (Mozobil) | Darapladib |
| | Floxuridine |
| | Fludarabine |

### Activity of Sole Compounds in a “Whole Virus” System

The inventors examined the activity of each of the chemicals in vitro by testing their ability to protect cells against viral death.

Cell were infected by the virus (1:100) and after 48 hours their viability was tested.

Without treatment roughly 50% of the cells died.

Results were compared relative to uninfected cells (100%), and to cells that received a drug “placebo” (0.1% DMSO).

Most compounds exhibited activity at varying extents. E inhibitors were found to have little/reduced activity at concentrations of 3 μM or lower (data not shown).

The two most effective compounds on their own were Darapladib and Flumatinib (which inhibit 3a).

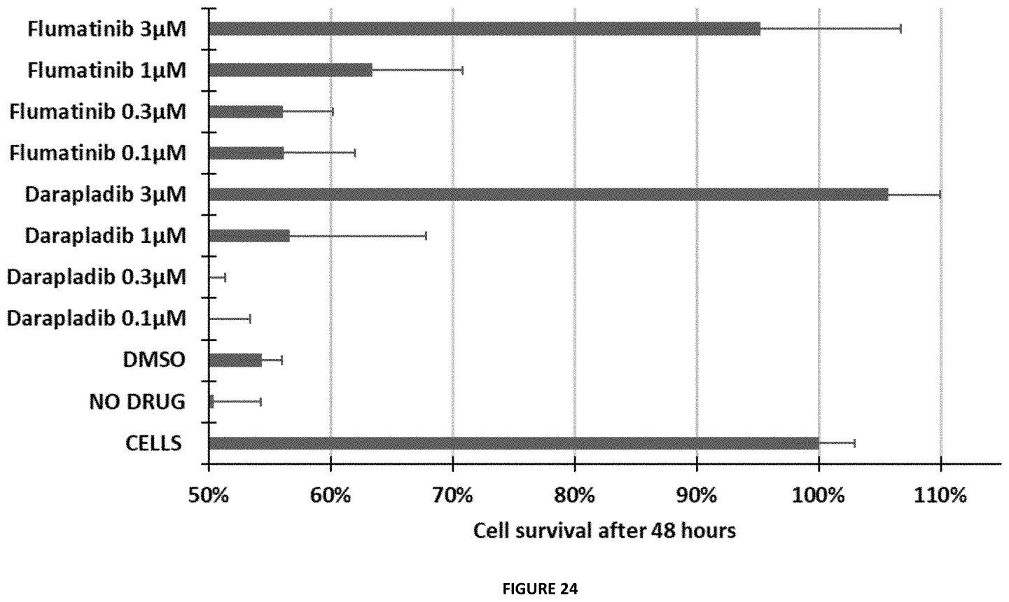

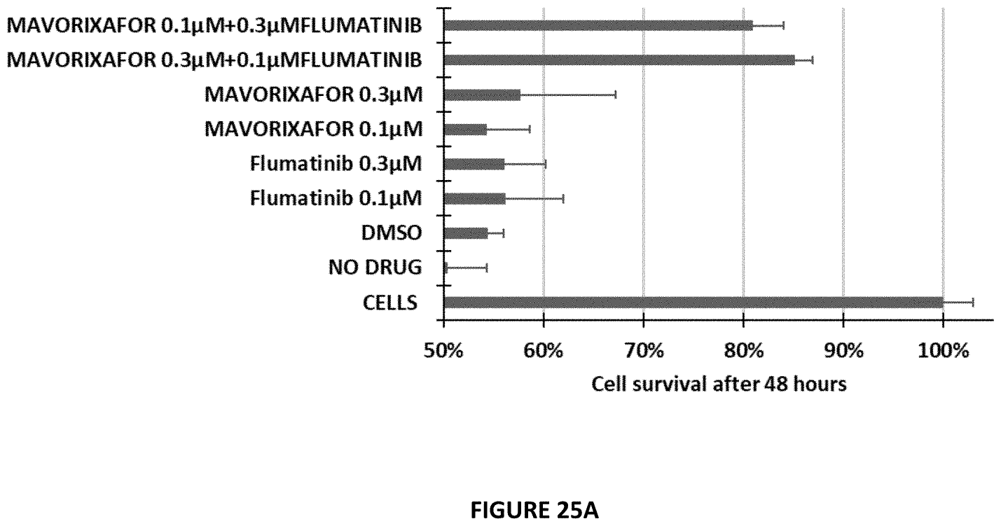

Specifically, the results show that in the presence of Flumatinib at concentrations of 0.1 to 3 approximately 60% or more cells have survived. In particular, Flumatinib at 3 provided near full survival (FIG. 24). Further, in the presence of Darapladib at concentrations of 1 to 3 approximately 60% or more cells have survived. In particular, Darapladib at 3 provided full survival (FIG. 24).

### Example 3

### Synergistic effects of E protein and 3a protein inhibitors

The inventors further sought to examine whether a combination of E inhibitors and 3a inhibitors would provide a better protection against viral-induced cell death.

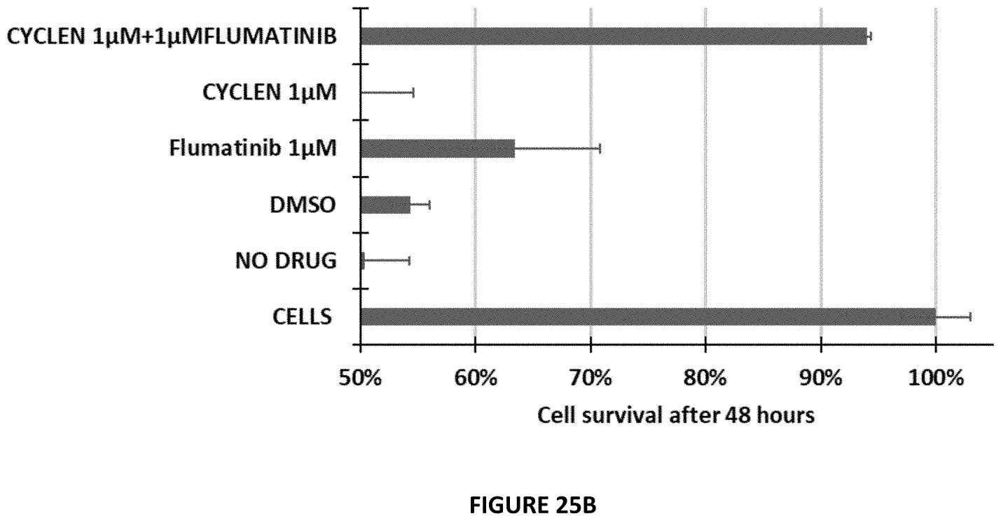

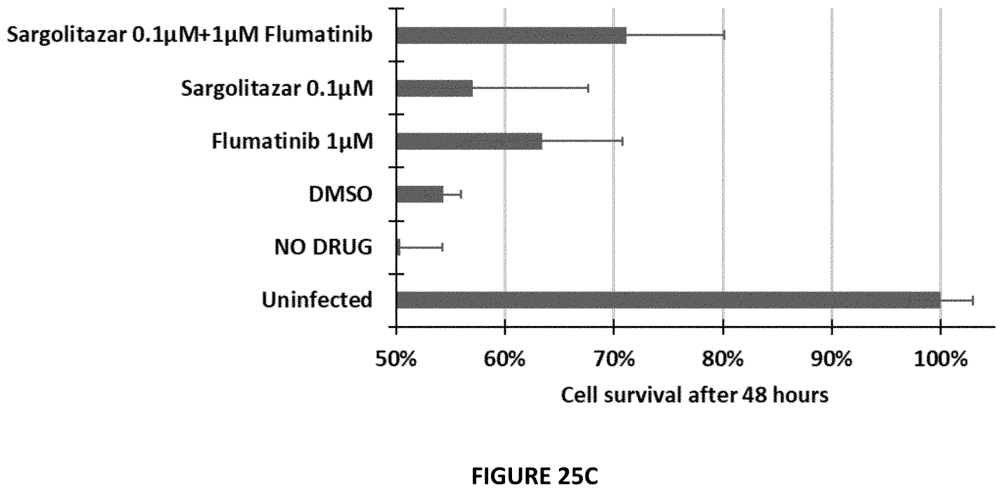

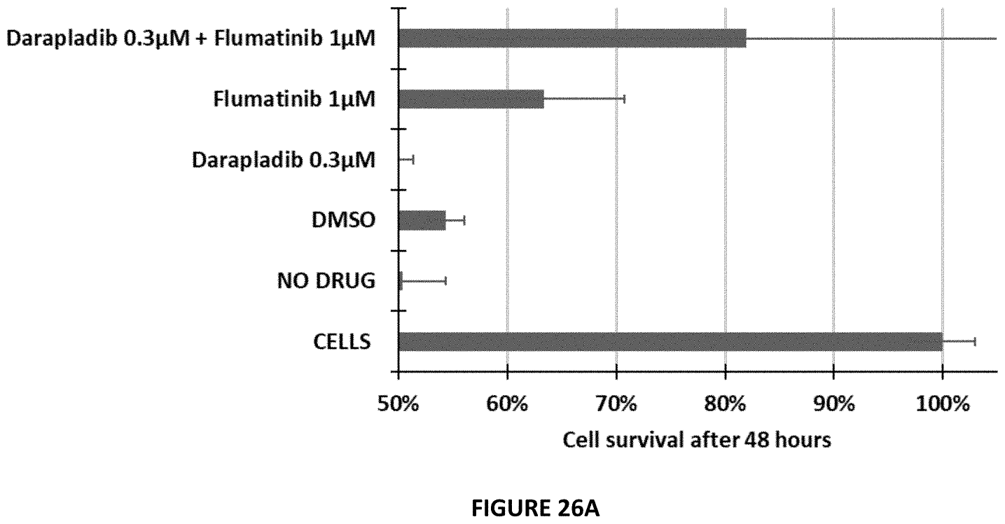

The inventors tested different combinations of Flumatinib (which inhibit 3a) with various E inhibitors (Mavorixafor, Cyclen, and Sargolitazar) at various combinations (0.1 μM-3 μM). The inventors showed particularly that Flumatinib and either Mavorixafor or Cyclen at a molar per molar ratio ranging from 3:1 to 1:3 results in a synergistic positive effect on cells survival, providing more than 80% viability (FIGS. 25A-25B). Specifically, Flumatinib and Mavorixafor at concentrations of either 0.1 μM and 0.3 μM or 0.3 μM and 0.1 μM provided the synergetic and increased cell viability effect (FIG. 25A). Further, Flumatinib and Cyclen at concentrations of 1 μM and 1 μM provided a synergetic effect, resulting with more than 90% cell viability (FIG. 25B).

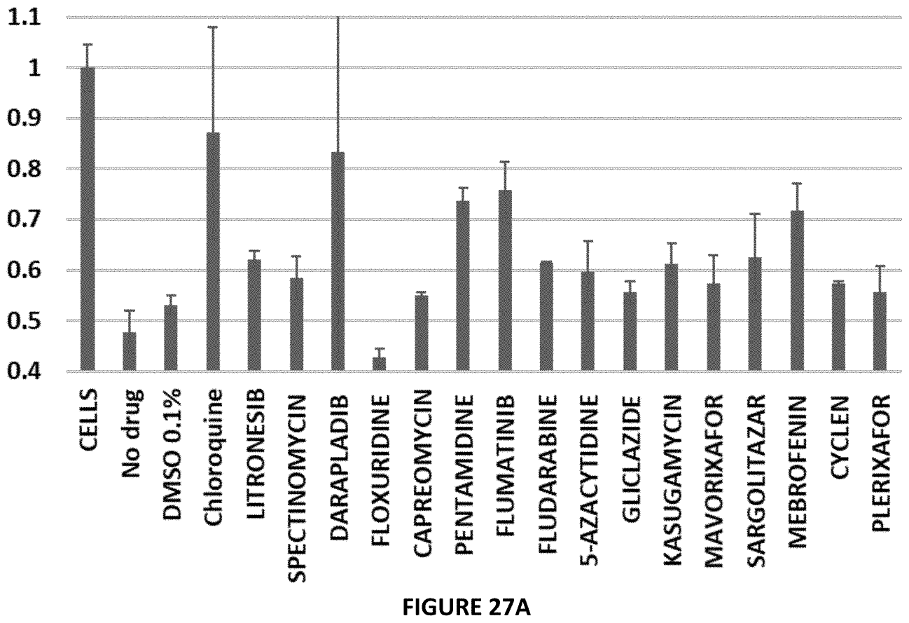

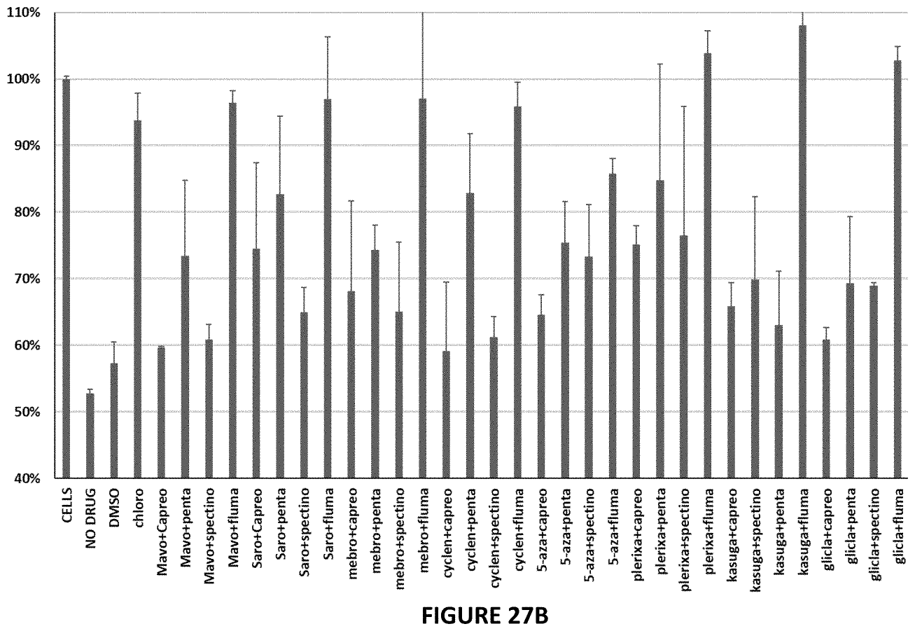

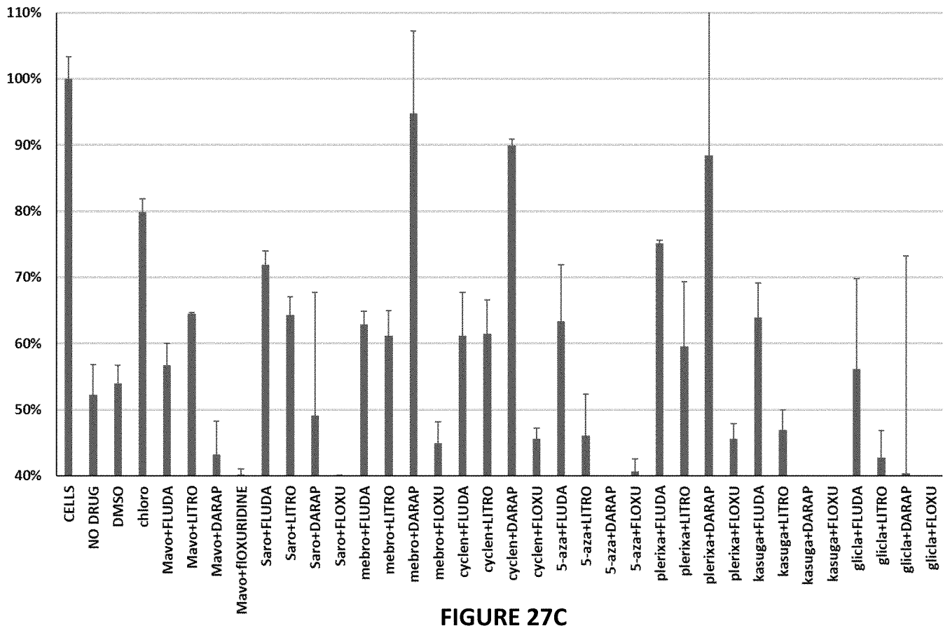

Further, the inventors have shown that at a signal concentration, the vast majority of E inhibitors positively reacted to a combination with Flumatinib (FIG. 27B). Further, the inventors showed that Darapladib exhibited a synergistic effect on the cell survival when provided along with Mavorixafor, Cyclen, and Plerixafor (FIG. 27C).

### Example 4

### Synergistic Effects of 3a Protein Inhibitors

The inventors further sought to examine whether a particular combination of 3a inhibitors would provide a better protection against viral-induced cell death.

The inventors tested different combinations of Flumatinib and Darapladib at various combinations (0.1 μM-3 μM).

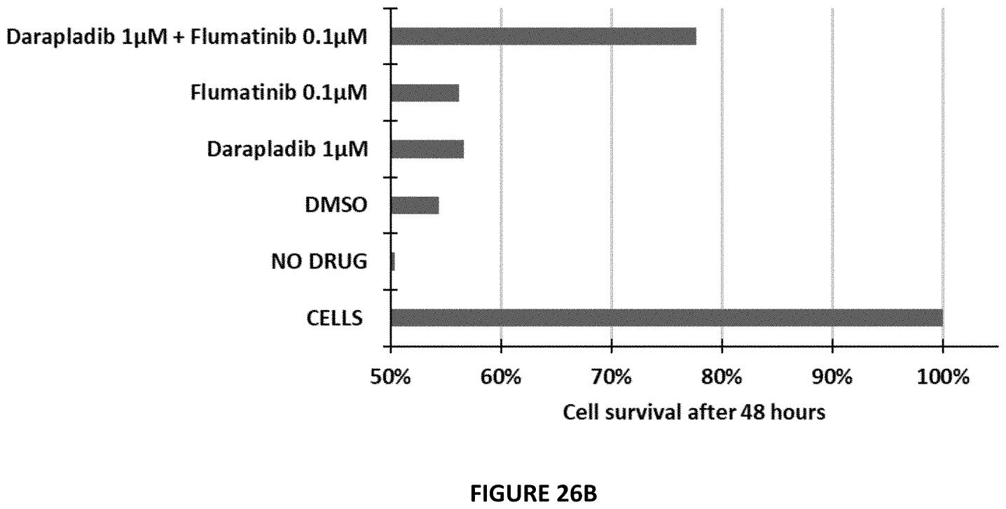

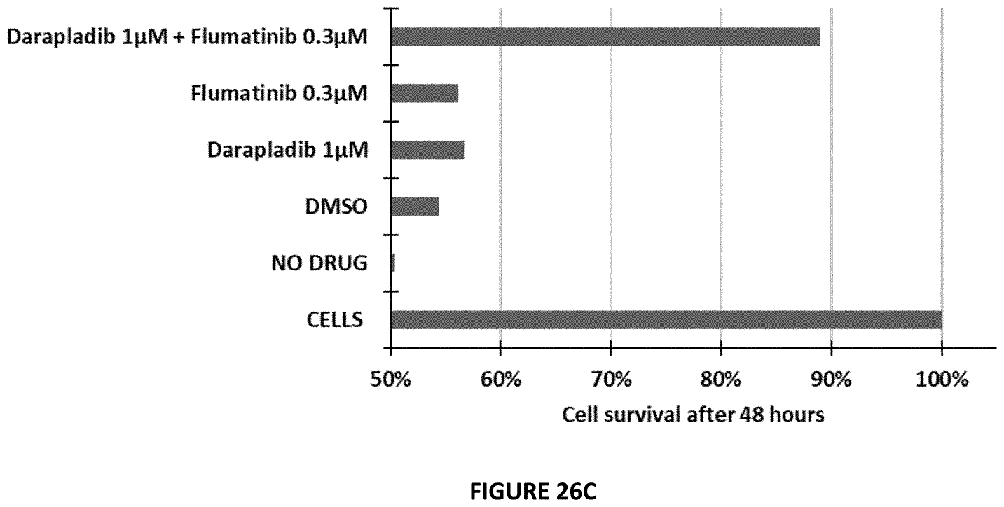

The inventors showed that Flumatinib and Darapladib at a molar per molar ratio ranging from 10:1 to 1:10 results in a synergistic positive effect on cells survival, providing approximately 80% viability, or more (FIGS. 26A-26C). Specifically, in the presence of Flumatinib and Darapladib at concentrations of either 1 μM and 0.3 μM or 0.1 μM and 1 respectively, approximately 80% of the cells survived (FIGS. 26A-26B). Further, Flumatinib and Darapladib at concentrations of 0.3 μM and 1 μM provided a synergetic effect, resulting with approximately 90% cell viability (FIG. 26C).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

What is claimed is:

1. A method for treating or preventing SARS-CoV-2 virulence in a subject in need thereof, comprising administering to said subject a therapeutically effective amount of any one of: SARS-CoV-2 E protein channel blocker and a SARS-CoV-2 3a protein inhibitor, thereby treating or preventing SARS-CoV-2 virulence in said subject.

2. The method of claim 1, wherein said preventing comprises preventing any one of: SARS-CoV-2 entry to a cell of said subject, uncoating of said SARS-CoV-2 in a cell of said subject, release of said SARS-CoV-2 from a cell of said subject, and any combination thereof.

3. The method of claim 1, wherein said subject is infected or suspected of being infected by SARS-CoV-2.

4. The method of claim 1, wherein said SARS-CoV-2 E protein channel blocker is at least one molecule selected from the group consisting of: 5-Azacytidine, Memantine, Gliclazide, Mavorixafor, Saroglitazar Magnesium, Mebrofenin, Cyclen, Kasugamycin, Plerixafor, and any salt thereof.

5. The method of claim 1, wherein said SARS-CoV-2 E protein channel blocker is for use at a daily dose of 0.01 to 500 mg/kg.

6. The method of claim 1, wherein said SARS-CoV-2 E protein channel blocker is Ginsenoside.

7. The method of claim 1, wherein said SARS-CoV-2 E protein channel blocker is Memantine.

8. The method of claim 1, wherein said SARS-CoV-2 3a protein inhibitor is at least one molecule selected from the group consisting of: Capreomycin, Pentamidine, Spectinomycin, Kasugamycin, Plerixafor, Flumatinib, Litronesib, Darapladib, Floxuridine, and Fludarabine.

9. The method of claim 8, wherein said SARS-CoV-2 3a protein inhibitor is Capreomycin.

10. A pharmaceutical composition comprising a SARS-CoV-2 3a protein inhibitor and a SARS-CoV-2 E channel blocker.

11. The pharmaceutical composition of claim 10, wherein said SARS-CoV-2 protein 3a inhibitor is selected from Flumatinib or Darapladib.

12. The pharmaceutical composition of claim 10, wherein said SARS-CoV-2 E channel blocker is selected from the group consisting of: Mavorixafor, Saroglitazar, Mebrofenin, Cyclen, Plerixafor, and Gliclazide.

13. The pharmaceutical composition of claim 10, wherein said SARS-CoV-2 protein 3a inhibitor is Flumatinib and said SARS-CoV-2 E channel blocker is selected from the group consisting of: Mavorixafor, Saroglitazar, Mebrofenin, Cyclen, Plerixafor, and Gliclazide.

14. The pharmaceutical composition of claim 10, wherein said SARS-CoV-2 protein 3a inhibitor is Darapladib and said SARS-CoV-2 E channel blocker is selected from the group consisting of: Mavorixafor, Mebrofenin, and Cyclen.

15. A method for treating or preventing SARS-CoV-2 virulence in a subject in need thereof, the method comprising administering to said subject a therapeutically effective amount of a pharmaceutical composition comprising a SARS-CoV-2 3a protein inhibitor and a SARS-CoV-2 E channel blocker, thereby treating or preventing SARS-CoV-2 virulence in said subject.

16. The method of claim 15, wherein said SARS-CoV-2 protein 3a inhibitor is selected from Flumatinib or Darapladib.

17. The method of claim 15, wherein said SARS-CoV-2 E channel blocker is selected from the group consisting of: Mavorixafor, Saroglitazar, Mebrofenin, Cyclen, Plerixafor, and Gliclazide.

18. The method of claim 15, wherein said SARS-CoV-2 protein 3a inhibitor is Flumatinib and said SARS-CoV-2 E channel blocker is selected from the group consisting of: Mavorixafor, Saroglitazar, Mebrofenin, Cyclen, Plerixafor, and Gliclazide.

19. The method of claim 15, wherein said SARS-CoV-2 protein 3a inhibitor is Darapladib and said SARS-CoV-2 E channel blocker is selected from the group consisting of: Mavorixafor, Mebrofenin, and Cyclen.


SEQUENCE LISTING (2 sequences)

SEQ ID NO: 1 - protein, 75 aa, unidentified
MYSFVSEETGTLIVNSVLLFLAFVVFLLVTLAILTALRLCAYCCNIVNVSLVKPSFYVYSRVKNLNSSRVPDLLV

SEQ ID NO: 2 - protein, 275 aa, Coronavirus
MDLFMRIFTIGTVTLKQGEIKDATPSDFVRATATIPIQASLPFGWLIVGVALLAVFQSASKIITLKKRWQLALSKGVHFVCNLLLLFVTVYSHLLLVAAGLEAPFLYLYALVYFLQSINFVRIIMRLWLCWKCRSKNPLLYDANYFLCWHTNCYDYCIPYNSVTSSIVITSGDGTTSPISEHDYQIGGYTEKWESGVKDCVVLHSYFTSDYYQLYSTQLSTDTGVEHVTFFIYNKIVDEPEEHVQIHTIDGSSGVVNPVMEPIYDEPTTTTSVPL